(19) United States
(12) Patent Application Publication
(10) Pub. No.: US 2009/0098540 A1
(43) Pub. Date: Apr. 16, 2009

(54) RECIRCULATING MICROFLUIDIC DEVICE AND METHODS OF USE

(75) Inventors: Antje J. Baeumner, Ithaca, NY (US); Kevin Nichols, Ithaca, NY (US); Sam R. Nugen, Ithaca, NY (US); Natalya V. Zaytseva, Ithaca, NY (US); Vasiliy N. Goral, Ithaca, NY (US); Sylvia Dokua Sakyiama Kwakye, Ithaca, NY (US)

Correspondence Address: NIXON PEABODY LLP - PATENT GROUP, 1100 CLINTON SQUARE, ROCHESTER, NY, 14604, US

(73) Assignee: Cornell Research Foundation, Inc, Ithaca, NY (US)

(21) Appl. No.: 11/916,921

(22) PCT Filed: Jun. 9, 2006
(86) PCT No.: PCT/US06/22638

§ 371 Date: Oct. 9, 2008

Related U.S. Application Data
(60) Provisional application No. 60/689,720, filed on Jun. 10, 2005.

(51) Int. Cl. C12Q 1/68; C12M 1/34

(52) U.S. Cl. 435/6; 435/287.2

(57) ABSTRACT

The present invention relates to a microfluidic test device for detecting or quantifying an analyte in a test sample. The device includes a non-absorbent substrate having at least one microchannel imbedded in the substrate, a non-specific capture device, and one or more stationary mixing structures extending into the at least one microchannel. The present invention also relates to relates to various methods of using the microfluidic test device to detect or quantify an analyte in a test sample. The present invention also relates to a microfluidic device that includes a non-absorbent substrate having at least one microchannel imbedded in the substrate and one or more stationary mixing structures extending into the at least one microchannel.

This application claims the benefit of U.S. Provisional Patent Application Ser. No. 60/689,720, filed Jun. 10, 2005, which is hereby incorporated by reference in its entirety.

The subject matter of this application was made with support from the United States Government under CSRESS Contract No. NYC-123-404 and National Institutes of Health Grant No. 1 R01 HD37109-01A1. The U.S. Government may have certain rights.

### FIELD OF THE INVENTION

The present invention is directed to a microfluidic device and to methods of using it.

### BACKGROUND OF THE INVENTION

Molecular biology based technologies, such as the polymerase chain reaction (PCR), for detection of pathogenic microorganisms are slowly replacing culture based detection methods (Kow et al., Journal of Medical Entomology 378(4):475-479 (2001); Laue et al., Journal of Clinical Microbiology 37(8):2543-2547 (1999); and Illen et al., Journal of Virol. Methods 41(2):135-146 (1993)). While molecular methods tend to be more sensitive, specific, and faster than culture based methods, they are also limited by expensive equipment requirements (Baeumner et al., Analytical Chemistry 74:1442-1448 (2002)). Scientists are overcoming this limitation by miniaturizing molecular assays into a microfluidic format (Mondesire et al., IVD Magazine 9-14 (2000); Yu et al., Micro Total Analysis Systems Conference, Enschede, Netherlands 545-548 (2000); Kopp et al., Science 280:1046-1048 (1998); and Manz et al., Journal of Chromatography 593:253-258 (1992)). Microfluidics is the enabling technology base for the development of miniature devices that move, mix, control, and react fluid volumes in the micron range. Microfluidics offer obvious advantages in the reduced consumption of reagents; faster and more sensitive reactions due to enhanced effects of processes such as diffusion and mass transport; increased throughput through parallel processing; and reduced expenses in terms of power and reagent consumption. Most importantly, fabrication of microfluidic devices is inexpensive and allows the integration of several modules to automate analytical processes (Duffy et al., Analytical Chemistry 70:4974-4984 (1998); Jingdong et al., Analytical Chemistry 72:1930-1933 (2000); and Martynova et al., Analytical Chemistry 69(23):4783-4789 (1997)).

A common feature of all nucleic acid detection methods in microarray chips and microchannels is the use of labels coupled to target specific probes. Typically, these labels are molecules that fluoresce, change, or produce color to indicate target hybridization to a probe (Ramsay, G., Nature Biotech 16:40-44 (1998)). Nanoparticles such as magnetic beads (Edelstein et al., Biosensors & Bioelectronics 14:805 (2000)), liposomes (Esch et al., Analytical Chemistry 73:2952-2958 (2001)) and gold particles (Taton et al., Science 289:1756-1760 (2002) and Cao et al., Science 297:1536-1540 (2002)) have also been used as labels. In most cases, these particle-labelled assays have proven to be more sensitive as they offer a means for further signal amplification that is not possible with conventional labels. Taton et al., for instance, use silver reduction to enhance visualization of gold particles in their assay (Taton et al., Science 289:1756-1760 (2002)). The least expensive and perhaps the simplest signal amplification scheme has been achieved with liposomes. Liposomes are phospholipid vesicles that entrap hundreds of thousands of marker molecules to provide a large signal amplification and enhanced sensitivity, 3 orders of magnitude greater than single fluorophore detection (Lee et al., Analytica Chimica Acta 354:23-28 (1997)).

Microfluidic mixers are an integral component of microscale total analysis systems (μTAS), which contain various modular units in a compact system (Manz et al., “Miniaturized total chemical analysis systems. A novel concept for chemical sensing,” Transducers '89: Proceedings of the 5th International Conference on Solid-State Sensors and Actuators and Eurosensors III. Part 1, Montreux, Switzerland (Jun. 25-30, 1989); van den Berg et al., Proceedings of the International Symposium on Micromechantronics and Human Science, pages 181-184 (1994); and Dhawan et al., Analytical and Bioanlytical Chemistry, 373:421-426 (2002)). Turbulence, the primary mechanism for macro-scale mixing, is effectively absent under normal conditions in most microfluidic systems due to low Reynolds numbers. Thus, alternative strategies for mixing in microfluidic systems must be employed. Several different strategies have been suggested in recent years based on a variety of different principles. Passive mixers use only the geometry of the channel to achieve mixing. Examples of passive mixers include those that generate transverse flows using a rigid arrangement of herring-bone structures to increase the interfacial area between liquids to be mixed (Stroock et al., Science 295:647-651 (2002)); that use a serpentine channel to simulate a partial packed bed of a chromatography column (He et al., “A Picoliter Volume Mixer for Microfluidic Analytical Systems,” Analytical Chemistry 73:1942 (2001)); and that use a T-junction mixer with deep well structures (Johnson et al., “Rapid Microfluidic Mixing,” Analytical Chemistry 74:45 (2002)). A thorough review of passive micromixers gives an overview of the basic physics involved in microscale mixing systems, and a discussion of the various geometries currently being employed in micromixers (Nguyen et al., “Micromixers—A review,” J. Micromech. Microeng. 15:R1-R16 (2005)). Active mixers generally use physical motion to induce mixing. An example of such a device is one that is based upon the movement of a stir bar under the influence of a magnetic field (Barbic et al., “Electromagnetic micromotor for microfluidics applications,” Applied Physics Letters 79:1399 (2001)). Another reported device includes a microfluidic device capable of recirculating nanoliter volumes within closed microfluidic channels, using counterbalancing hydrodynamic pressure against an electro-osmotically generated flow in a dead-end chamber (Lammertink et al., Anal. Chem. 76:3018-3022 (2004)). However, there is a need for a microfluidic mixer that does not have the deficiencies (noted above).

The present invention is directed to overcoming the above deficiencies in the art.

### SUMMARY OF THE INVENTION

The present invention relates to a microfluidic test device for detecting or quantifying an analyte in a test sample. The device includes a non-absorbent substrate having at least one inlet and outlet extending therethrough. The inlet and outlet are connected by at least one microchannel imbedded in the substrate. The at least one microchannel includes an inlet portion and an analysis portion. The device also includes a non-specific capture device located at or upstream of the analysis portion. The device further includes one or more stationary mixing structures extending into the at least one microchannel.

The present invention also relates to a method for detecting or quantifying an analyte in a test sample. This involves providing at least one test mixture where the test mixture includes a test sample, potentially containing analyte, a capture conjugate, and a marker conjugate. The capture conjugate includes a capture support and a first binding material, where the first binding material is selected to bind with a portion of the analyte. The marker conjugate includes a particle, a marker, and a second binding material, where the second binding material is selected to bind with a portion of the analyte other than the portion of the analyte for which the first binding material is selected. The method also involves providing a microfluidic test device for detecting or quantifying an analyte in a test sample. The test device includes a non-absorbent substrate having at least one inlet and outlet extending therethrough, where the inlet and outlet are connected by at least one microchannel imbedded in the substrate, and where the at least one microchannel includes an inlet portion and an analysis portion. The test device also includes a non-specific capture device located at or upstream of the analysis portion. The test device further includes one or more stationary mixing structures extending into the at least one microchannel. Reaction is permitted to occur, within the microfluidic test device, in the test mixture between analyte present in the test sample and the first and second binding materials, thereby forming a product complex that includes analyte present in the test sample, the capture conjugate, and the marker conjugate. The reacted test mixture is contacted to the non-specific capture device (e.g., a device having non-specific affinity for the capture support), whereby product complex present in the reacted test mixture is immobilized from the reacted test mixture. The presence or amount of the marker from the immobilized product complex is detected at the analysis portion of the microfluidic test device and correlated with the presence or amount, respectively, of the analyte in the test sample.

Another aspect of the present invention relates to a method for detecting or quantifying an analyte in a test sample, as follows: This method includes providing at least one test mixture which includes a test sample potentially containing an analyte, a capture support complex including a capture support and a first member of a first coupling group, a first binding material selected to bind with a portion of the analyte and including a second member of the first coupling group, a marker complex which includes a particle, a marker, and a first member of a second coupling group, and a second binding material selected to bind with a portion of the analyte other than the portion of the analyte for which the first binding material is selected and including a second member of the second coupling group. The method also involves providing a microfluidic test device for detecting or quantifying an analyte in a test sample. The test device includes a non-absorbent substrate having at least one inlet and outlet extending therethrough, where the inlet and outlet are connected by at least one microchannel imbedded in the substrate, and where the at least one microchannel includes an inlet portion and an analysis portion. The test device also includes a non-specific capture device located at or upstream of the analysis portion. The test device further includes one or more stationary mixing structures extending into the at least one microchannel. Reaction is permitted to occur, within the microfluidic test device, in the at least one test mixture between the first and second members of the first coupling group, between the first and second members of the second coupling group, and between analyte present in the test sample and the first and second binding materials. As a result, a product complex including analyte present in the test sample, the capture support complex, the first binding material, the marker conjugate, and the second binding material is formed. The reacted test mixture is contacted to a non-specific capture device (e.g., a device having non-specific affinity for the capture support) so that product complex present in the reacted test mixture is immobilized from the reacted test mixture. The presence or amount of the marker from the immobilized product complex is detected at the analysis portion of the microfluidic test device and correlated with the presence or amount, respectively, of the analyte in the test sample.

Another aspect of the present invention relates to a method for detecting or quantifying an analyte in a test sample, as follows: This involves providing at least one test mixture including a test sample potentially containing an analyte, a capture conjugate (including a capture support and a first binding material), where the first binding material is selected to bind with a portion of the analyte, and a marker conjugate (including a particle, a marker, and an analyte analog). The method also involves providing a microfluidic test device for detecting or quantifying an analyte in a test sample. The test device includes a non-absorbent substrate having at least one inlet and outlet extending therethrough, where the inlet and outlet are connected by at least one microchannel imbedded in the substrate, and where the at least one microchannel includes an inlet portion and an analysis portion. The test device also includes a non-specific capture device located at or upstream of the analysis portion. The test device further includes one or more stationary mixing structures extending into the at least one microchannel. Competition is permitted to occur, within the microfluidic test device, in the at least one test mixture between analyte present in the test sample and the analyte analog for the first binding material. As a result, a product complex, including the capture conjugate and the marker conjugate, is formed. The reacted test mixture is contacted to a non-specific capture device (e.g., a device having non-specific affinity for the capture support) so that product complex present in the reacted test mixture is immobilized from the reacted test mixture. The immobilized product complex is detected at the analysis portion. The presence or amount of the marker from the immobilized product complex is correlated with the presence or amount, respectively, of the analyte in the test sample.

The present invention also relates to a microfluidic device (also referred to herein as a recirculating microfluidic device, a microfluidic mixing device, or the like). This device includes a non-absorbent substrate having at least one inlet and outlet extending therethrough and one or more stationary mixing structures. The at least one inlet and outlet are connected by at least one microchannel imbedded in the substrate. The one or more stationary mixing structures extend into the at least one microchannel.

Microfluidics combined with a liposome signal amplification scheme, in accordance with the present invention, promises an inexpensive solution to the heightened need for technology that can rapidly and accurately detect pathogenic organisms in environmental, clinical, and food samples in the wake of recent threats of bioterrorism. Liposome technology has been used in analogous membrane detection systems with great success (Baeumner et al., Analytical Chemistry 74:1442-1448 (2002); Esch et al., Analytical Chemistry 73:3162-3167 (2001); and Rule et al., Clinical Chemistry 42:206-1209 (1996), which are hereby included by reference in their entirety). It has been reported that gains in sensitivity can be achieved by converting a liposome-based membrane detection assay for Cryptosporidium parvum to a microfluidic format (Esch et al., Analytical Chemistry 73:3162-3167 (2001); and Rule et al., Clinical Chemistry 42:206-1209 (1996); and Taton et al., Science 289:1756-1760 (2002), which are hereby incorporated by reference in its entirety) (see also Goral et al., “Electrochemical microfluidic biosensor for the detection of nucleic acid sequences,’ Lab on a Chip 6(6):414-421 (2006); Zaytseva et al., “Microfluidic biosensor for the serotype-specific detection of dengue virus RNA,” Analytical Chemistry 77(23):7520-7527 (2005); and Zaytseva et al., “Development of a microfluidic biosensor module for pathogen detection,” Lab on a Chip 5(8):805-811 (2005), which are hereby incorporated by reference in their entirety).

The passive microfluidic mixer of the present invention is capable of establishing a recirculating flow inside mobile and open volumes from the nanoliter to the microliter range. Mixing in the device occurs not by generating transverse flows perpendicular to the length of the channel (see Stroock et al., Science 295:647-651 (2002), which is hereby incorporated by reference in its entirety), but instead by generating transverse flows parallel to the length of the channel, such that streamline segments at different lengths of the channel can be brought into contact with each other. It takes advantage of a fluid-exchange principle (described in U.S. Pat. No. 6,331,073 to Chung et al., which is hereby incorporated by reference in its entirety): the device provides order-changing functions to a microfluid, i.e., allowing sections of fluid separated by a length of the channel to interact directly. The device effectively “folds” the solution to permit streamlines that are normally linearly separated to come into contact. In one embodiment, the microfluidic device is a microfluidic mixer that is pressure driven in an open-end chamber using an attached syringe controlled by an external motor.

The present invention relating to the recirculating microfluidic mixer can find application in a variety of bioanalytical and chemical micro/nano systems, such as (but not limited to) microfluidic sensors, micro-Total Analysis Systems. For example, it can be used for the effective and rapid mixing of several solutions, it can be used to decrease the time needed for a nucleic acid sequence-based amplification (NASBA) reaction, or any catalytically derived reaction, any hybridization reaction, any binding reaction (e.g., RNA-DNA hybridization reactions using liposome and magnetic beads with immobilized DNA oligonucleotides).

### BRIEF DESCRIPTION OF THE DRAWINGS

FIG. 1 shows the principle scheme of the biosensor of the present invention based on DNA/RNA hybridization.

FIGS. 2A-B show the fabrication of the polydimethylsiloxane (“PDMS”) microchannels using a silicon wafer as mold and a second silicon wafer as a lid to control flatness and thickness of the PDMS layer. FIG. 2A shows these components in an arrangement where the PDMS layer is between the first and second silicon wafers, as would be the case where the PDMS layer is being formed. FIG. 2B shows an exploded view where the first silicon wafer, the PDMS layer, and second silicon wafer are separated from one another after the PDMS layer is formed.

FIGS. 3A-B show the channel layouts for fluorescence and electrochemical detections. FIG. 3A shows the channel layout for fluorescent method of signal detection. The enlarged area 106 of the channel near outlet 110 is the detection zone, and the channel with inlet 102 is the main hybridization channel.

FIG. 3B shows the channel layout for an electrochemical method of signal detection. The wider channel 106 between where inlet 102 and inlet 108 merge and where the outlet 110 represents the detection zone.

FIG. 4 shows the assembly of the microfluidic channel device 20. PDMS layer 22 with the microchannels 24 is laid onto a glass plate 26 in order to provide a cover for the channel structure.

FIG. 5 shows the assembly of microfluidic device 32 in a housing. The PDMS 22-glass plate 26 structure is held together by applying slight pressure using a housing that consists of two plates 28 and 4-8 screws 30.

FIGS. 6A-B show interdigitated ultramicroelectrode arrays (“IDUA”) in accordance with the present invention.

FIG. 7 shows a device for fluorescent detection in accordance with the present invention.

FIG. 8 shows the positioning of a magnet in a capture zone of a two-channel microfluidic device in accordance with the present invention. Fluorescence microscopy was used to measure the fluorescence in the detection zone (capture zone) on top of the magnet. While other fluorescence detection devices can be used, a fluorescence microscope was chosen here in order to observe the different steps in the analysis optically while the reactions were occurring.

FIG. 9 shows a device for electrochemical detection in accordance with the present invention.

FIG. 10 shows the positioning of a PDMS microchannel layer on top of an IDUA transducer on the glass plate in accordance with the present invention.

FIG. 11 shows a simplified block diagram of the analysis system instrumentation.

FIG. 12 shows the original potentiostat circuit. The IDUA potential is set with a 1.2V voltage reference (Vref) and adjusted with a 1 MΩ potentiometer. The sensor output is first converted to a voltage, amplified, and output to an LCD or a data-logger connected to a computer. The gain of the current-to-voltage amplifier is adjusted with the switch S2.

FIG. 13 shows the sensor output versus time for detection of 0.1M of potassium ferri- and ferrohexacyanide. Channel 1 graphs the bias potential held constant at 400 mV, while channel 2 is the current-to-voltage amplifier output in mV.

FIG. 14A shows the dose response curve for ferri/ferrohexacyanide detection. FIG. 14B shows an expanded view of FIG. 14A for concentrations of 0, 0.1, and 1 μM.

FIG. 15 shows a microcontroller program flow. Operation is interrupt driven where the microcontroller (“MCU”) stays in low power mode until an interrupt occurs. It then enters active mode and performs the event requested by the interrupt.

FIG. 16 shows the fluorescence images of the captured superparamagnetic beads with no RNA is in the sample (background) and when there is a complex with target RNA and bound nonlysed (A)/lysed liposomes (B).

FIG. 17 shows the fluorescence intensity vs. amount of liposomes.

FIG. 18 shows the fluorescence intensity vs. amount of magnetic bead.

FIG. 19 shows the standard curve for the determination of lower limit of detection. Error bars correspond to 3×Standard Deviations.

FIG. 20 shows the IDUA's response in the microchannel upon the injection of 20 nL, 50 nL, 100 nL of 10 μM Fe²⁺/Fe³⁺. Buffer flow rate is 1 μL/min. Buffer background signal—0.27±0.01 nA, 20 nL signal—0.95±0.03 nA, 50 nL signal—2.06±0.05 nA, 100 nL signal—4.15±0.1 nA.

FIG. 21 shows the signal response of an IDUA in the microchannel in the presence and in the absence of an analyzed RNA.

FIGS. 22A-B are schematics showing the structure of the microfluidic mixer device. The top image (FIG. 22A) shows the design of one sawtooth unit. The bottom image (FIG. 22B) is a cross section of the device. The gray areas indicate the PDMS device itself, and the white areas are the housing made of PMMA. The holes in the top PMMA and PDMS layers are used to allow access to the channel, which is molded into the bottom PDMS layer. The PMMA layers are used for structural support and for macroscopic interconnect placement (not shown).

FIG. 23 shows a two-dimensional velocity profile, viewed from above. The top image is of left to right flow, and the bottom image is of right to left flow. The length of the microchannel shown is 150 μm. The magnitude of velocity is coded in colors as per the scale on the left side [m/s].

FIG. 24 are velocity profiles for left to right flow along the length of one sawtooth unit of three streamlines. Velocities are given on the y-axis in m/s. The “top” streamline is that which begins 37.5 μm=(0.75*50 μm) from the lower wall in FIG. 2 (the streamlines are all 2D functions that are in the plane of the paper). The “middle” streamline is that which begins 25 μm from both the top and bottom wall. The “bottom” stream line is that which begins 12.5 μm from the bottom wall.

FIG. 25 are velocity profiles for right to left flow along the length of one sawtooth unit of three streamlines. Velocities are given on the y-axis in m/s. The “top” streamline is that which begins 37.5 μm=(0.75*50 μm) from the lower wall in FIG. 2 (the streamlines are all 2D functions that are in the plane of the paper). The “middle” streamline is that which begins 25 μm from both the top and bottom wall. The “bottom” stream line is that which begins 12.5 μm from the bottom wall.

FIG. 26 shows a time lapse image of Labeled DMSO/Unlabeled Hydrocarbon Plug/Labeled DMSO moving right (first four frames) and left (bottom seven frames) over four sawtooth units.

FIGS. 27A-B show a photograph and illustration of four separate volumetric elements recirculated next to each other. Rapid back and forth cycling was manually performed to obtain this image. A channel was filled with one half fluorescently labeled DMSO, and one half unlabeled DMSO and rapidly mixed with reciprocating flows. Four regions of clearly varying concentration are visible in the photograph (FIG. 27A) and illustrated below (FIG. 27B).

FIG. 28 shows a configuration of microchannels. The “Inlet 1” ports are the main inlets for the reaction solutions. “Inlet 2” is used as the surfactant inlet for liposome lysis. Both inlets used the same outlet which is located at the end of the detection zone.

FIG. 29 shows dimensions of the sawtoothed microchannel. All measurements are in microns. Each microdevice contains 20 columns (5 cm) each with 166 sawteeth.

FIG. 30 is a diagram representing the channels used for the mixing studies. For the purposes of the study, a length of 0 was set where the channel width became 50 μm.

FIG. 31 shows a cross section of a sawtoothed channel. The pixel intensity was measured in a line extending through the cross section midway between two sawteeth.

FIG. 32 shows a comparison of mixing profiles between a sawtoothed channel and a straight channel. Pixel intensity profiles were taken at the midpoint between sawteeth and the equivalent distance in the smooth channel. Length of 0 designates the point that the inlets inter the channel. The DI water inlet was on side indicated as channel width 50, and the 50 mM fluorescein at channel width 0.

FIG. 33 shows the standard deviation of the pixel intensities at various lengths of the channel. The sawtooth channel (--) appears to reach a smaller standard deviation over the given length verses the straight channel (-∘-).

FIGS. 34A-B show a schematic (FIG. 34A) representation of IDUA and optical photographs (FIG. 34B) of IDUA at 1.25×, 5× and 20× magnification (see Goral et al., “Electrochemical microfluidic biosensor for the detection of nucleic acid sequences,’ Lab on a Chip 6(6):414-421 (2006), which is hereby incorporated by reference in its entirety).

FIG. 35 is a schematic showing various embodiments of microchannels having one or more stationary mixing structures 300 extending into the microchannel 200. This figure shows the top view of channels (for example in PDMS 800) (through the open face channels showing the sawtooth structures).

FIGS. 36A-C are schematics showing the microchannels (e.g., PDMS microchannels) with sawtooth structures. FIG. 36A shows a side view of a packaged device of the present invention. Key: Microchannel 400; Glass slide cover 500; Housing 600 (e.g., acrylic housing); and Screws 700. FIG. 36B shows a side view of channels (e.g., in PDMS). Key: PDMS 800; and Microchannel 400. FIG. 36C shows a top view of channels in PDMS (through the open face channels showing the sawtooth structures 900). Key: PDMS 800; and Microchannel 400. Arrows indicate fluid flow through device.

FIG. 37 is a schematic showing the top view of channels in PDMS 800 with two inlet channels 890 (through the open face channels showing the sawtooth structures 900).

### DETAILED DESCRIPTION OF THE INVENTION

The present invention relates to a microfluidic test device for detecting or quantifying an analyte in a test sample. The device includes a non-absorbent substrate having at least one inlet and outlet extending therethrough. The inlet and outlet are connected by at least one microchannel imbedded in the substrate. The at least one microchannel includes an inlet portion and an analysis portion. The device also includes a non-specific capture device located at or upstream of the analysis portion. The device further includes one or more stationary mixing structures extending into the at least one microchannel.

In one embodiment, there are a plurality of stationary mixing structures extending into the at least one microchannel. These stationary mixing structures can extend different lengths into the at least one microchannel. In another embodiment, each microchannel has opposite sides with at least some of the stationary mixing structures extending into the microchannel from the opposite sides in directions generally toward one another. In a further embodiment, the microfluidic test device can include one or more stationary mixing structures that extend into the one or more microchannels at an inclined angle. There can be a plurality of the stationary mixing structures with at least some extending into the one or more microchannels at different angles. In still another embodiment, there can be a plurality of inlets to each microchannel.

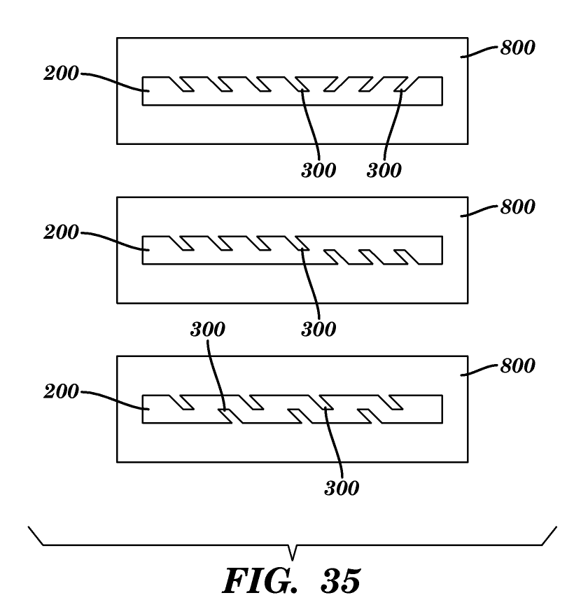

As used herein, the term stationary mixing structure can also be referred to as a sawtooth. The sawteeth can be of variable length (in order to catch different streamlines). The sawteeth can have variable angles. The sawtooth arrangement can included reversing sawteeth (i.e., having one set of sawteeth as shown plus a set mirrored to is in a second part of the channel), or putting sawteeth on either or both sides of the channel walls. Examples are shown in FIG. 35.

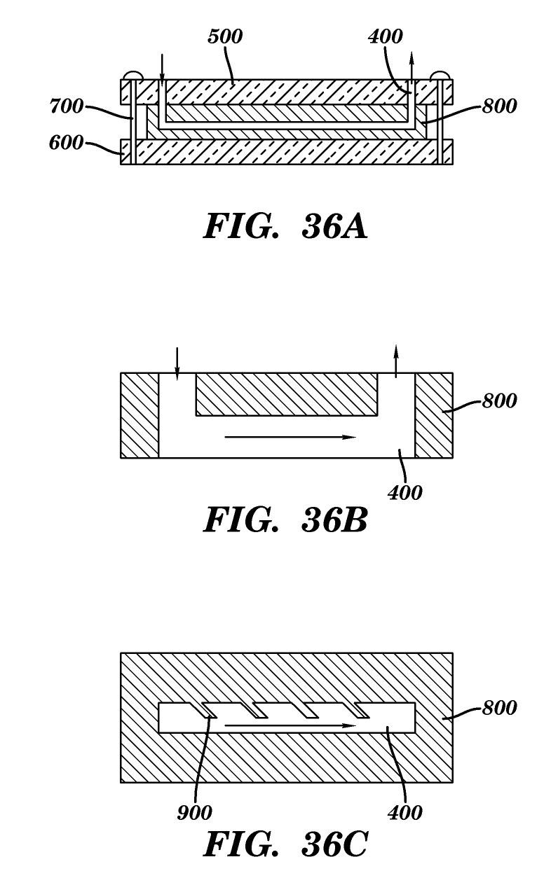

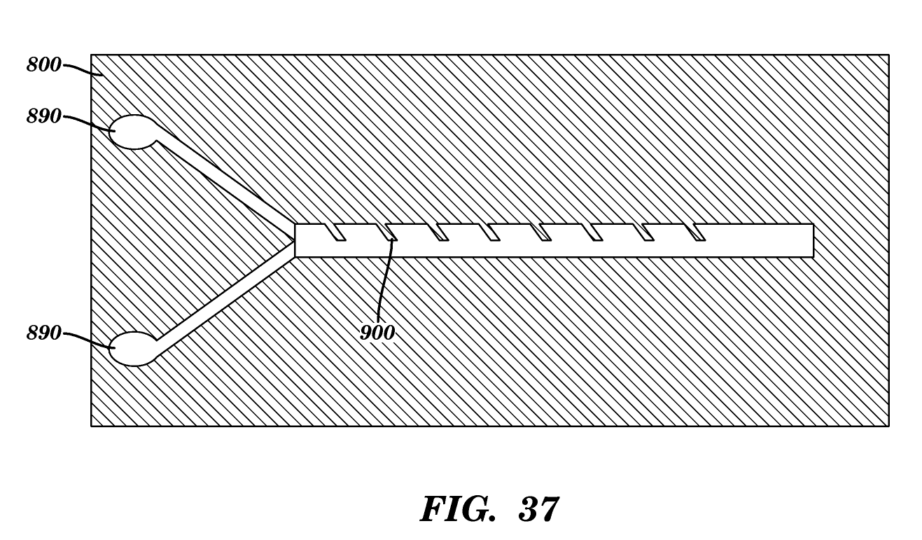

The channel length is not critical and will simply provide more or less volume. Thus, devices have been fabricated that can have 5 nL total volume and those that can have 15 nL volume (as an example). The mixer can be made of other materials than PDMS, including (but not limited to) Si, SiO2, SU8, quartz, acrylic, etc. (see FIG. 36). Mixing can be carried out for any fluid that may also contain particulates or larger molecules, including, without limitation, liposomes, magnetic beads, cells, nucleic acids, enzymes, etc. The design can be used for a NASBA reaction (nucleic acid sequence-based amplification) and for the liposome assays. In yet another embodiment, the microfluidic device includes two inlet channels leading into the main sawtooth channel (see FIG. 37).

In one embodiment, the microfluidic device is capable of recirculating microliter volumes is described. The device consists of molded polydimethyl siloxane (PDMS) channels with pressure inlet and outlet holes sealed by a glass lid (see FIG. 36). Recirculation is accomplished by repeatedly changing the direction of flow over an iterated sawtooth structure. The sawtooth structure serves to change the fluid velocity of individual streamlines differently dependent on whether the fluid is flowing backwards or forwards over the structure. In this manner, individual streamlines can be accelerated or decelerated relative to the other streamlines to allow sections of the fluid to interact that would normally be linearly separated. Low Reynolds numbers imply that the process is reversible, neglecting diffusion. Fluorescent indicators were employed to verify numerical simulations. It was found that mixing of a Carboxyfluorescein labeled DMSO plug with an unlabeled DMSO plug across an immiscible hydrocarbon plug reached steady state in the channels with the sawtooth structures after 7.1 min, versus 34.8 min in the channels without sawtooth structures, which verified what would be expected based on numerical simulations.

The recirculating microfluidic mixer of the present invention can be used in a variety of bioanalytical and chemical micro/nano systems, such a (but not limited to) microfluidic sensors, micro-Total Analysis Systems. For example, it can be used for the effective and rapid mixing of several solutions, it can be used to decrease the time needed for a nucleic acid sequence-based amplification (NASBA) reaction, or any catalytically derived reaction, any hybridization reaction, any binding reaction.

The microfluidic device can also be used in an NASBA reaction, and subsequently also for RNA-DNA hybridization reactions using liposome and magnetic beads with immobilized DNA oligonucleotides.

The non-absorbent substrate is formed from a material like quartz, glass, polymethylacrylate, polydimethyl siloxane, or polymeric materials.

The microfluidic test device can additionally include an incubation portion upstream of the analysis portion.

When the capture device and the analysis portion are at the same location, the complex containing analyte, capture conjugate, and marker conjugate can be detected at the capture device. When the analysis portion is downstream of the capture device, marker is released from the complex immobilized to the capture device and detected as it moves with fluid in the direction from inlet 102 to outlet 110. In a third embodiment, the analysis portion is located upstream of the capture device so that when marker is released from the immobilized complex, it is carried to the analysis portion by then reversing flow of fluid in the direction from outlet 110 to inlet 102.

The electrochemical detection assembly comprises a microcontroller-based analysis system. An example of such a system is described in the following paragraphs.

The current instrumentation is at once a potentiostat for electrochemical detection, a data acquisition/storage system, and a controller for the active components (such as the pump actuator and electromagnet) of the microfluidic biosensor. Requirements for portability, low power consumption, and a small form factor are achieved with an electronic design that uses as few components as possible.

The heart of the system is the low power, highly integrated MSP430FG439 microcontroller (“MCU”) from Texas Instruments. Texas Instruments produces a large range of devices that differ only in terms of the number of I/O pins, integrated peripherals, memory, and price. The underlying architecture of all the MCUs are the same. Thus, code written for one MCU will work on all MCUs with a few changes to the initialization setup. The flexibility offered by MCU choice allows the manufacture of inexpensive basic analysis systems as well as deluxe systems using the same code-base. Furthermore, the system can be easily upgraded with an advanced MCU.

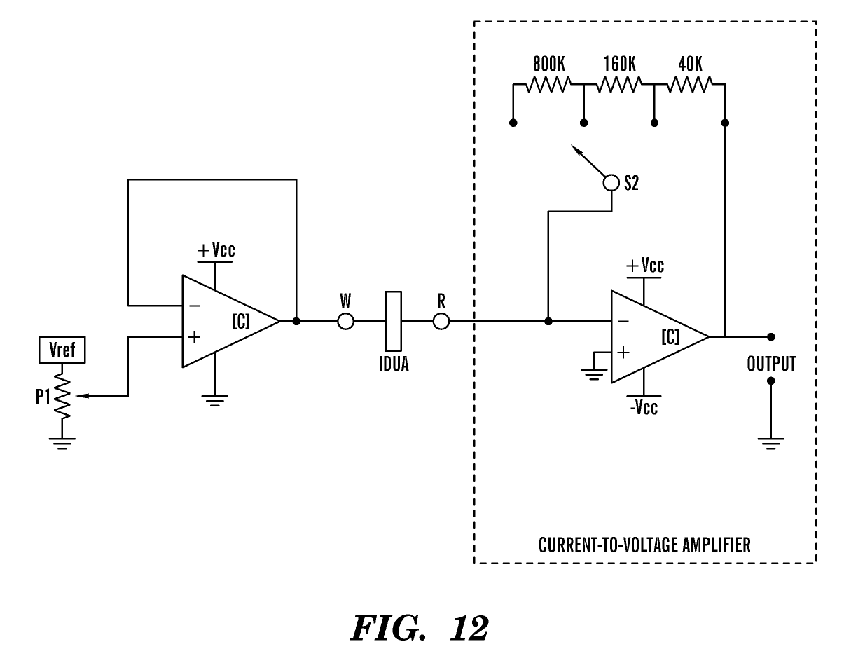

The MSP430 has 4 main sections —CPU, memory, clock, and peripherals. See FIG. 11. The CPU performs all the calculations and data manipulation.

The MSP430FG439 has 60 KB of program memory and 2K SRAM. Program memory is flash and self-programmable. This feature allows about 100 data files to be stored for 1-minute measurements taken in 1 sec intervals. The storage capacity can be increased with extra non-volatile memory modules.

The clock system is very flexible and allows the device to operate in a very low power mode at 32 KHz for unattended periodic measurements for instance, and up to a fast 8 MHz for data acquisition, analysis, transmission, and display in real time.

Most of the system's functionality is provided by the MCU's peripherals. The MSP430FG439 has a built in liquid crystal display (“LCD”) controller, 1 universal synchronous asynchronous receiver transceiver (“USART”), an 8 channel 12-bit analogue-to-digital-conversion (“ADC”) port, 2 channel 12 bit digital-to-analogue conversion (“DAC”) port, 3 operational amplifiers, a built in supply voltage supervisor, 6 general input/output (I/O) ports and 4 timers. In this application, the basic timer is used to maintain a real time clock and time stamps for logged data. It also supplies the LCD frame frequency rate. Timer A is used to generate alarm and distinctive status beeps on a buzzer. Timer B is used to generate PWM outputs used to control peripherals external to the MCU. Any of the timers can be set to keep track of the interval and duration of measurement. The watchdog timer can also reset the device when errors occur during operation.

The firmware for the MCU is written mainly in C and compiled with the open-source MSPGCC compiler for the MSP430 line of microcontrollers. The microcontroller is in-circuit programmable via a JTAG interface. The current design calls for the JTAG headers to be left in the circuit so that the firmware can be upgraded and easily debugged. However, the interface can also be removed to prevent tampering. Writing the code in C offers another distinctive advantage to this system, with the addition of a hardware configuration file for the parts and peripherals, any capable microcontroller can be substituted for the MSP430 line of microcontrollers.

The other major components of the system are the analogue chain couplings to the ADC and DAC channels of the MCU. Each ADC channel is coupled to a programmable gain current-to-voltage amplifier. The amplifier converts the current induced in an IDUA sensor to a voltage and amplifies it. The signal is then captured and logged by the analogue-to-digital converter of the MCU. The potential signal is converted back to current in software before display.

The built-in DAC peripheral supplies a bias potential of up to 2.5V for the IDUA. The potential is adjusted by a user via the user interface described in more detail below.

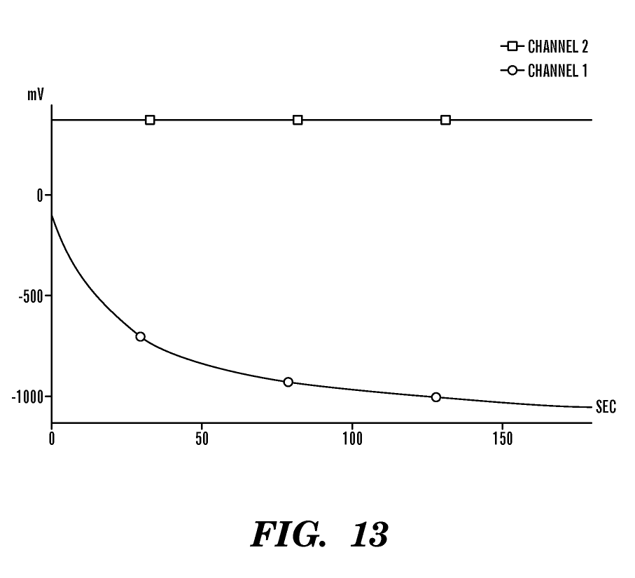

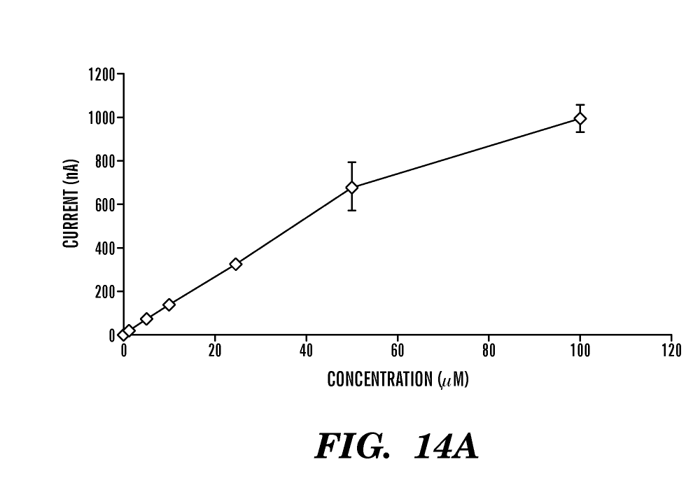

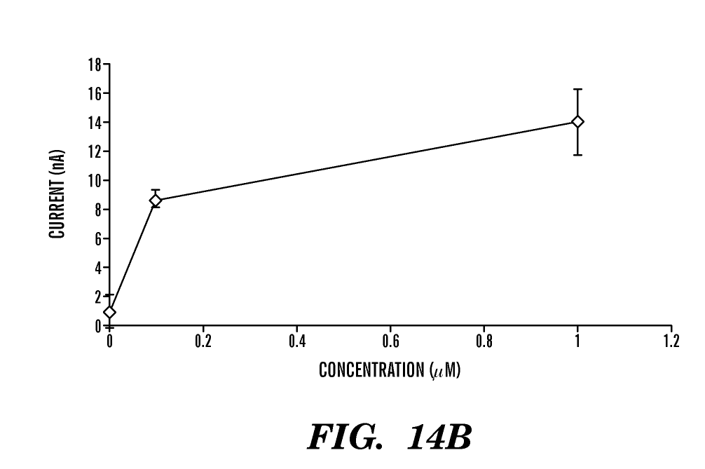

The ADC and DAC analogue chains form a potentiostat for the electrochemical detection scheme of the biosensor. As mentioned earlier, this circuit was derived from a standalone analogue version that was thoroughly tested. FIGS. 12, 13, 14A, and 14B show the original potentiostat circuitry and the results of electrochemical detection of the redox pair potassium ferri/ferrohexacyanide on a gold IDUA at a potential of 400 mV. The IDUA had 400 fingers. The fingers were on average 1000 A high and 2 μm wide with 0.9 μm gaps size between them. The resistance of the current-to-voltage amplifier was set to 200 KΩ (sensor current voltage/200000). Unless specified, measurements were taken at 1 second intervals for a duration of 1 minute.

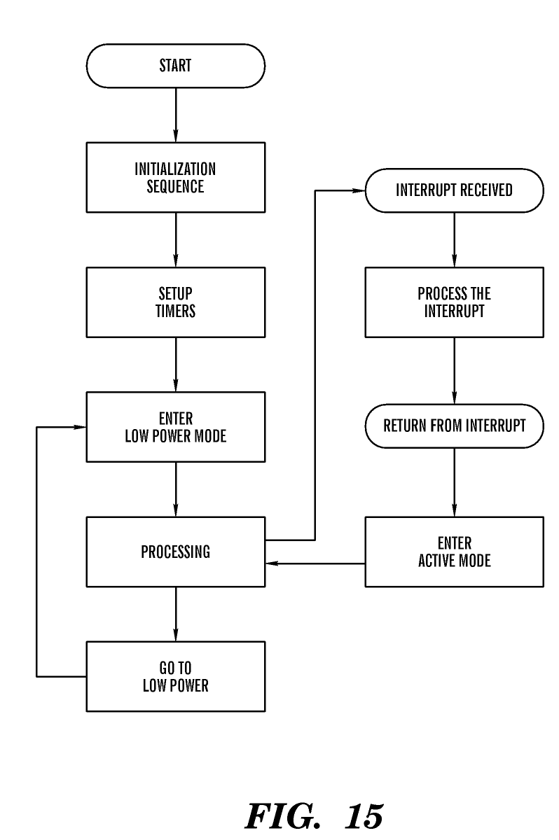

The operation of the microcontroller-based device is interrupt driven. For the most part, the MCU stays in low power mode. In this mode, the real time clock is on but most of the peripherals are turned off. The device enters active mode only in response to interrupts generated by pressing one of the push buttons; communication received on the USART; a power-on reset; low battery alarm; or any of the timers. The operations are summarized in the FIG. 15.

The device is battery powered. When connected, a power-on reset starts up the MCU. It goes through an initialization sequence and preps its peripherals and timers. The MCU then enters and stays in the main loop of low power mode with bursts of activity generated by other interrupts.

Every interrupt received is processed in order of priority. Each interrupt wakes the MCU up and puts it in active mode to perform whatever activity is required. Once all the instructions have been processed in active mode, the MCU goes back into low power mode to wait for the next event.

Four push buttons generate interrupts that turn the LCD display on or off, initiate measurements, initiate a change in the parameters, and puts the device in monitor mode where the device wakes up periodically to take a measurement at predetermined intervals. The functions are not fixed and can be re-programmed as needed.

The device also wakes up when it receives an input on its USART port. This input may be a request to retrieve logged data for instance. A low battery interrupt disables most of the MCU activity and generates alarms that may include—beeping and/or flashing the low battery sign on the LCD. The watchdog timer generates an interrupt if there is a problem with the execution n of an instruction. This interrupt will cause the device to re-initialize itself with default parameters and notify the user accordingly.

The user interface currently includes an LCD, a serial connection to a computer, 4 buttons as well as connections to a keypad. The interface also includes a cross-platform graphical user interface (“GUI”) with access to the underlying platform's internet capabilities that a client may use to change measurement or control parameters, upload/download data, and visualize sensor output. The modular design of the system allows other communication schemes such as ethernet, infrared, and wireless to be easily integrated as needed.

The GUI provides an easy to use menu-driven interface for adjusting sensor potential, full scale measurement range, measurement interval, communication settings and setting the correct time. Currently, sensor potential may range from 0 to 1500 mV. Full scale range (+/−) may be 10 nA to 1 mA. Measurement interval is a minimum of 0.5 seconds at the moment.

There is no restriction on measurement duration if data storage is not required of the MCU. The capacity of the MCU at the moment is restricted to 6000 data points. Duration thus depends on capacity. Thus, for 1 sec intervals, measurement duration should not exceed 100 mins. Capacity can be increased to 30000 data points in the MCU's flash memory. Also, as mentioned earlier, capacity can be increased with external dataflash.

The GUI also allows the user to watch sensor signals change in real time on a graph or graph data downloaded from the MCU. The data can also be saved as comma delimited files for viewing and analysis in third party applications.

The present invention also relates to a method for detecting or quantifying an analyte in a test sample. This involves providing at least one test mixture where the test mixture includes a test sample, potentially containing analyte, a capture conjugate, and a marker conjugate. The capture conjugate includes a capture support and a first binding material, where the first binding material is selected to bind with a portion of the analyte. The marker conjugate includes a particle, a marker, and a second binding material, where the second binding material is selected to bind with a portion of the analyte other than the portion of the analyte for which the first binding material is selected. The method also involves providing a microfluidic test device for detecting or quantifying an analyte in a test sample. The test device includes a non-absorbent substrate having at least one inlet and outlet extending therethrough, where the inlet and outlet are connected by at least one microchannel imbedded in the substrate, and where the at least one microchannel includes an inlet portion and an analysis portion. The test device also includes a non-specific capture device located at or upstream of the analysis portion. The test device further includes one or more stationary mixing structures extending into the at least one microchannel. Reaction is permitted to occur, within the microfluidic test device, in the test mixture between analyte present in the test sample and the first and second binding materials, thereby forming a product complex that includes analyte present in the test sample, the capture conjugate, and the marker conjugate. The reacted test mixture is contacted to the non-specific capture device (e.g., a device having non-specific affinity for the capture support), whereby product complex present in the reacted test mixture is immobilized from the reacted test mixture. The presence or amount of the marker from the immobilized product complex is detected at the analysis portion of the microfluidic test device and correlated with the presence or amount, respectively, of the analyte in the test sample. In one embodiment, the permitting reaction to occur and the contacting steps are carried out by cycling the test mixture in opposite directions in the at least one microchannel.

The term “analyte” is meant to include the compound or composition to be measured or detected. It is capable of binding to the first and second binding materials. Suitable analytes include, but are not limited to, antigens (e.g., protein antigens), haptens, cells, and target nucleic acid molecules. A preferred analyte is a target nucleic acid molecule. The present invention is applicable to procedures and products for determining a wide variety of analytes. As representative examples of types of analytes, there may be mentioned: environmental and food contaminants, including pesticides and toxic industrial chemicals; drugs, including therapeutic drugs and drugs of abuse; hormones, vitamins, proteins, including enzymes, receptors, and antibodies of all classes; prions; peptides; steroids; bacteria; fungi; viruses; parasites; components or products of bacteria, fungi, viruses, or parasites; aptamers; allergens of all types; products or components of normal or malignant cells; etc. As particular examples, there may be mentioned T₄; T₃; digoxin; hCG; insulin; theophylline; leutinizing hormones; and organisms causing or associated with various disease states, such as Streptococcus pyrogenes (group A), Herpes Simplex I and II, cytomegalovirus, chlamydiae, etc. The invention may also be used to determine relative antibody affinities, and for relative nucleic acid hybridization experiments, restriction enzyme assay with nucleic acids, binding of proteins or other material to nucleic acids, and detection of any nucleic acid sequence in any organism, i.e., prokaryotes and eukaryotes. A more preferred analyte is a target nucleic acid molecule found in an organism selected from the group consisting of bacteria, fungi, yeast, viruses, protozoa, parasites, animals (e.g., humans), and plants. Suitable organisms include, but are not limited to, Cryptosporidium parvum, Escherichia coli, Bacillus anthracis, Dengue virus, and Human immunodeficiency virus (HIV-1).

The term “binding material” is meant to include a bioreceptor molecule such as an immunoglobulin or derivative or fragment thereof having an area on the surface or in a cavity which specifically binds to and is thereby defined as complementary with a particular spatial and polar organization of another molecule—in this case, the analyte. Suitable binding materials include antibodies, antigens, nucleic acid molecules, aptamers, cell receptors, biotin, streptavidin, and other suitable ligands. When the analyte is a target nucleic acid molecule, the first binding material can be a nucleic acid molecule (e.g., reporter probe, selected to hybridize with a portion of the target nucleic acid molecule) and the second binding material can be a nucleic acid molecule (e.g., capture probe, selected to hybridize with a separate portion of the target nucleic acid molecule), or other moiety, such as an antibody or other agent capable of binding to and interacting with the analyte.

Antibody binding materials can be monoclonal, polyclonal, or genetically engineered (e.g., single-chain antibodies, catalytic antibodies) and can be prepared by techniques that are well known in the art, such as immunization of a host and collection of sera, hybrid cell line technology, or by genetic engineering. The binding material may also be any naturally occurring or synthetic compound that specifically binds the analyte of interest.

The first and second binding materials are selected to bind specifically to separate portions of the analyte. For example, when the analyte is a nucleic acid sequence, it is necessary to choose probes for separate portions of the target nucleic acid sequence. Techniques for designing such probes are well-known. Probes suitable for the practice of the present invention must be complementary to the target analyte sequence, i.e., capable of hybridizing to the target, and should be highly specific for the target analyte. The probes are preferably between 17 and 25 nucleotides long, to provide the requisite specificity, while avoiding unduly long hybridization times and minimizing the potential for formation of secondary structures under the assay conditions. Thus, in this embodiment, the first binding material is reporter probe, which is selected to, and does, hybridize with a portion of target nucleic acid sequence. The second binding material, referred to herein as a capture probe for the nucleic acid detection/measurement embodiment, is selected to, and does, hybridize with a portion of target nucleic acid sequence other than that portion of the target with which reporter probe hybridizes. The capture probe may be immobilized in a capture portion of the microchannel or on a magnetic bead. In addition, the first and second binding materials (reporter and capture probes) should be capable of no or limited interaction with one another. Techniques for identifying probes and reaction conditions suitable for the practice of the invention are described in Sambrook et al., Molecular Cloning: A Laboratory Manual, Cold Spring Harbor Laboratory Press (1989), which is hereby incorporated by reference in its entirety. A software program known as “Lasergene”, available from DNASTAR, or similar products may optionally be used.

The method of the invention employs marker complexes which include a particle, a marker, and one member of a coupling group. Suitable particles include liposomes (the marker may be encapsulated within the liposome, or incorporated in the bilayer), latex beads, gold particles, silica particles, dendrimers, quantum dots, magnetic beads (e.g., antibody-tagged magnetic beads and nucleic acid probe-tagged magnetic beads), or any other particle suitable for derivatization. Where multiple marker complexes are used, the marker in each complex may be the same or different.

The use of liposomes as described in the present application provides several advantages over traditional signal production systems employing, for example, enzymes. These advantages include increased signal intensity, shelf stability, and instantaneous release of signal-producing markers, as described in Siebert et al., Analytica Chimica Acta 282:297-305 (1993); Yap et al., Analytical Chemistry 63:2007 (1991); Plant et al., Analytical Biochemistry 176:420-426 (1989); Locascio-Brown et al., Analytical Chemistry 62:2587-2593 (1990); and Durst et al., Eds., Flow Injection Analysis Based on Enzymes or Antibodies, vol. 14, VCH, Weinheim (1990), each of which is hereby incorporated by reference in its entirety.

Liposomes can be prepared from a wide variety of lipids, including phospholipids, glycolipids, steroids, relatively long chain alkyl esters; e.g., alkyl phosphates, fatty acid esters; e.g. lecithin, fatty amines, and the like. A mixture of fatty materials may be employed, such as a combination of neutral steroid, a charge amphiphile and a phospholipid. Illustrative examples of phospholipids include lecithin, sphingomyelin, and dipalmitoylphosphatidylcholine, etc. Representative steroids include cholesterol, chlorestanol, lanosterol, and the like. Representative charge amphiphilic compounds generally contain from 12 to 30 carbon atoms. Mono- or dialkyl phosphate esters, or alkylamines; e.g. dicetyl phosphate, stearyl amine, hexadecyl amine, dilaurylphosphate, and the like are representative.

The liposome vesicles are prepared in aqueous solution containing the marker, whereby the vesicles will include the marker in their interiors. The liposome vesicles may be prepared by vigorous agitation in the solution, followed by removal of the unencapsulated marker. Alternatively, reverse phase evaporation plus sonication can be used. Further details with respect to the preparation of liposomes are set forth in U.S. Pat. No. 4,342,826 and PCT International Publication No. WO 80/01515, both of which are hereby incorporated by reference in their entirety.

The concentration of electrolytes in the medium will usually be adjusted to achieve isotonicity or equi-osmolality (or up to about 50 to about 100 mmol/kg hypertonic) with the solution in the interior of liposomes to prevent their crenation or swelling.

With some increased complexity of the excitation waveform applied by the electroanalyzer, electrochemical measurement in accordance with the invention may also be carried out using stripping voltammetry, employing, for example, liposome encapsulated metal ions for detection and measurement.

Moderate, and desirably substantially constant, temperatures are normally employed for carrying out the assay. The temperatures for the assay and production of a detectable signal will generally be in the range of about 4-65° C., more usually in the range of about 20-38° C., and frequently, will be about 15-45° C.

The solvent for the test mixture will normally be an aqueous medium, which may be up to about 60 weight percent of other polar solvents, particularly solvents having from 1 to 6, more usually of from 1 to 4, carbon atoms, including alcohols, formamide, dimethylformamide and dimethylsulfoxide, dioxane, and the like. Usually, the cosolvents will be present in less than about 30-40 weight percent. Under some circumstances, depending on the nature of the sample, some or all of the aqueous medium could be provided by the sample itself.

The pH for the medium will usually be in the range of 2-11, usually 5-9, and preferably in the range of about 6-8. The pH is chosen to maintain a significant level of binding affinity of the binding members and optimal generation of signal by the signal producing system. Various buffers may be used to achieve the desired pH and maintain the pH during the assay. Illustrative buffers include borate, phosphate, carbonate, tris, barbital, and the like. The particular buffer employed is usually not critical, but in individual assays, one buffer may be preferred over another. For nucleic acid analytes, it is necessary to choose suitable buffers. Such buffers include SSC, sodium chloride, sodium citrate buffer, and SSPE (sodium chloride, sodium phosphate, EDTA).

This method can be carried out with the bioanalytical microsystem which includes a sample preparation module and a biosensor module. This entire system is preferably produced in a microfluidic platform.

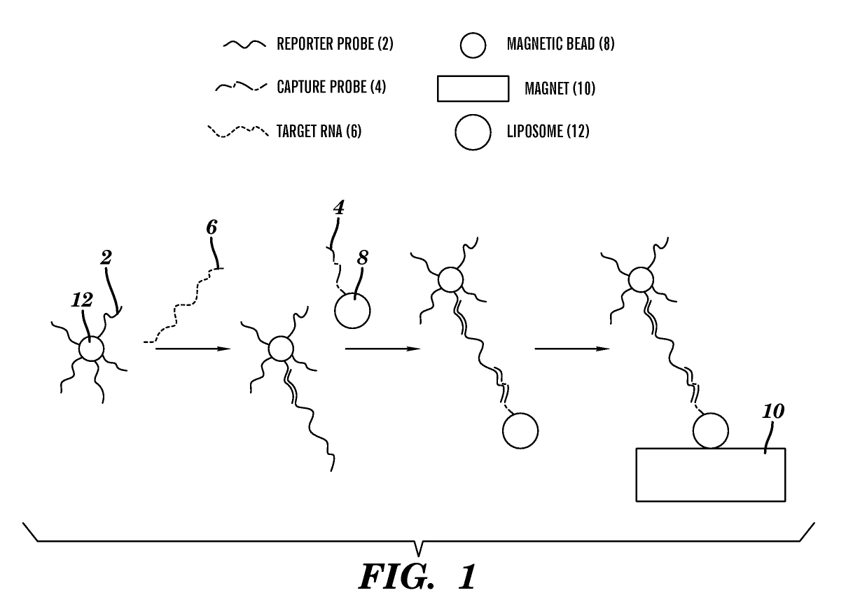

The principle of the biosensor of the present invention is based on DNA/RNA hybridization system and liposome signal amplification (FIG. 1). As shown in FIG. 1, two sets of probes hybridize specifically with the target RNA. This system is described with reference to a generic probe designed to bind to four Dengue virus serotypes, and four specific probes are designed to bind to the four serotypes only (Wu et al., “Detection of Dengue Viral RNA Using a Nucleic Acid Sequence-based Amplification Assay,” J. Clin. Microbiol. 39:2794-2798 (2001), which is hereby incorporated by reference in its entirety). The reporter probe is coupled to liposomes with an encapsulated fluorescent dye or an electrochemically active compound and can hybridize to a specific sequence of the target RNA. The second specific probes (capture probes) are immobilized on the surface of superparamagnetic microbeads via biotin-streptavidin interaction. Target RNA is amplified using the isothermal nucleic-acid-sequence-based amplification (“NASBA”) reaction. Liposomes with reporter probes and beads with capture probes are incubated with amplified target sequence prior to introducing the mixture into the microchannel where the sandwich complexes subsequently are captured on the magnet for fluorescence or electrochemical detection.

The microfluidic device of the present invention can be designed to carry out fluorescent or electrochemical methods for signal detection. The approach for construction of the microfluidic device was based on providing precise sample handling in terms of volume and flow-rates, zero-dead volume at inlet and outlet points (no sample losses during the analysis and 100% waste disposal), ability to disassemble the device for replacing the microfluidic channel or transducer parts. Liposome, Dengue virus RNA, reporter and capture probes, and hybridization and washing buffers were used as optimized in experiments previously carried out in the development of membrane strip-based biosensors for Dengue virus detection (Baeumner et al., “A Biosensor for Dengue Virus Detection: Sensitive, Rapid and Serotype Specific,” Analytical Chemistry, 74(6):1442-1448 (2002) and Zaytseva et al., “Multi-Analyte Single-Membrane Biosensor for Serotype-Specific Detection of Dengue Virus,” Anal. Bioanal. Chem. 380:46-53 (2004), which are hereby incorporated by reference in their entirety).

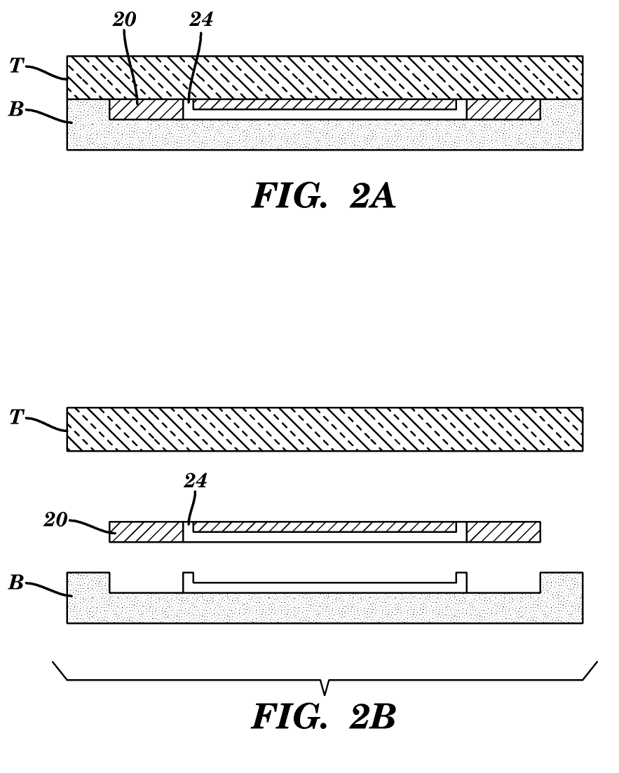

Microfluidic channels can be fabricated as raised structures on 4 inch silicone wafers using standard photolithography processes. A 1 mL of freshly prepared 7:1 by volume mixture of silicone elastomer and silicone elastomer curing agent (Sylgard, 184 Silicone elastomer kit) was poured onto the silicone template and covered with another flat silicone wafer. Covering the resulting polydimethylsiloxane (PDMS) layer with a silicone wafer allowed thickness and thickness uniformity of the layer to be controlled. The obtained sandwich structure (FIG. 2) was cured in an oven at 65° C. for 2 h. The cured PDMS layer was peeled off the wafer and channels were manually cut out. The PDMS layer was 170 micrometer thick. The channels were 50 mm deep and the width was varied from 100 to 500 micrometer.

Microfluidic channels employed in the microfluidic device of the present invention should fulfill the following requirements: 1) The geometry and dimensions of a channel should be suitable to avoid large pressure drops in the liquid flow upon entering it. Experimentally, it was found that 100 micrometer wide, 50 micrometer deep channel satisfied this requirement; 2) The channel should have a small region with 5 times slower linear fluid flow compared to the rest of it. In this region, magnetic beads that are utilized during the analysis are captured. The signal transducer is placed downstream of the captured beads area; and 3) The PDMS layer with embedded channels structure should have inlet and outlet holes that go vertically through the entire width of the PDMS and that have a diameter not bigger than the width of the adjacent channel. These appropriate inlet dimensions are especially needed in order to avoid volumes of stagnated flow during the analysis.

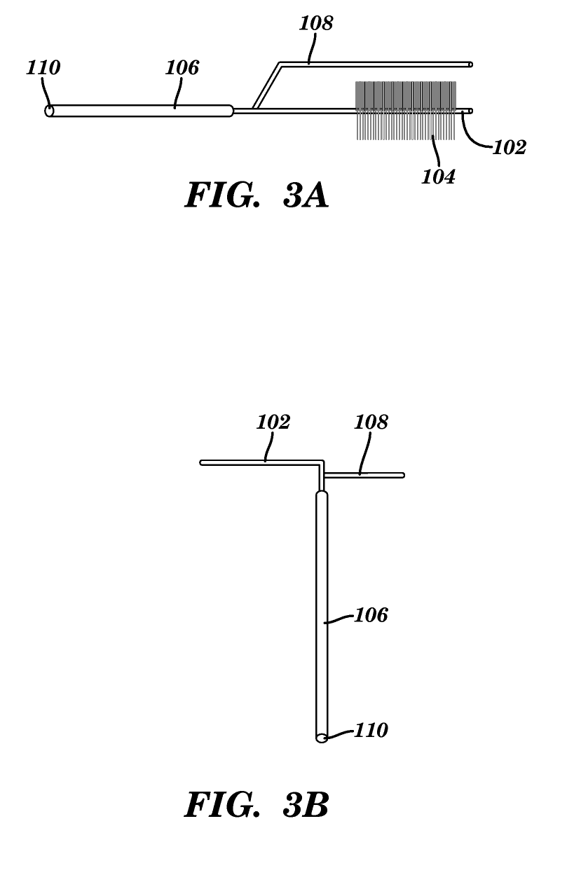

Typical channel geometries with dimensions in micrometers are presented in FIG. 3. FIGS. 3A and 3B show 2 embodiments of the present invention where each has inlets 102 and 108 leading to detection section 106 and ultimately to outlet 110. In FIG. 3A, inlet 102 has a circuitous region 104 which gives the materials passing through more residence time to undergo reaction.

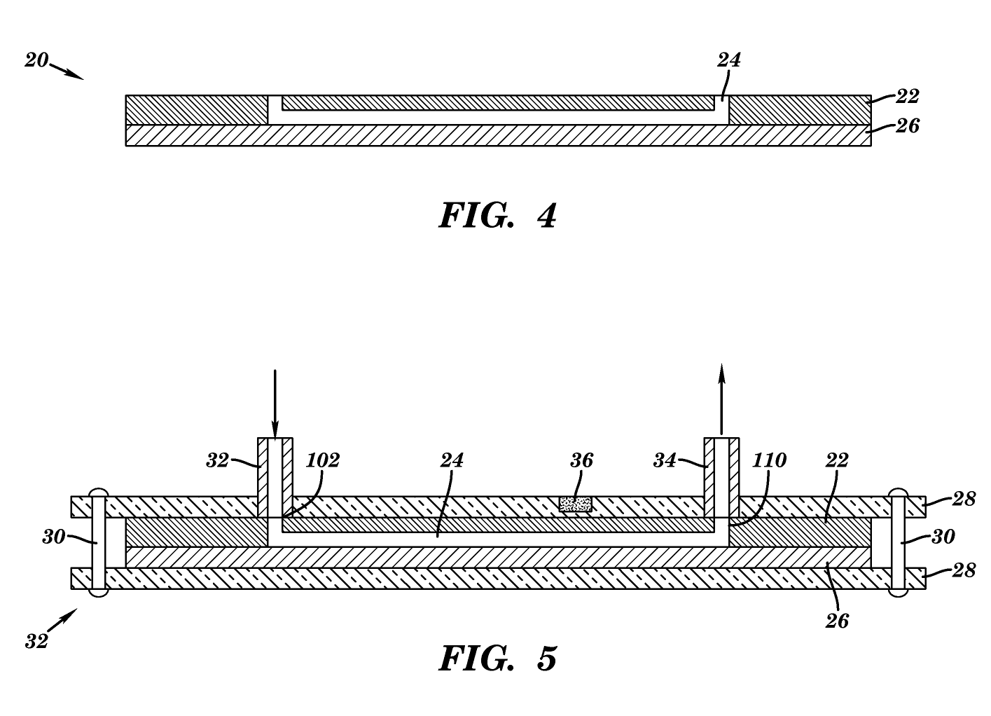

The formation of the PDMS 22 with embedded channels 24 is shown in FIGS. 2A to 2B. As shown in FIG. 2A, PDMS 22 with embedded channels 24 is formed by molding between top mold plate T and bottom mold plate B. PDMS 22 is formed with embedded channels 24, which are longitudinally exposed along one surface of PDMS 22. This structure is recovered by removing it from mold plates T and B, as shown in FIG. 2B. PDMS 22 with embedded channels 24 is mounted to glass plate 26 to form unit 20, as shown in FIG. 4. Such mounting causes the longitudinally exposed channels 24 in PDMS to be covered by glass plate 26.

For the microfluidic device with fluorescent detection, glass plate 26 is clear. In the case of electrochemical detection, glass plate 26 is provided with patterned interdigitated ultramicroelectrode arrays (IDUAs) which are in fluid communication with channels 24 so that material passing through channel 24 can be in contact with and analyzed by the IDUAs.

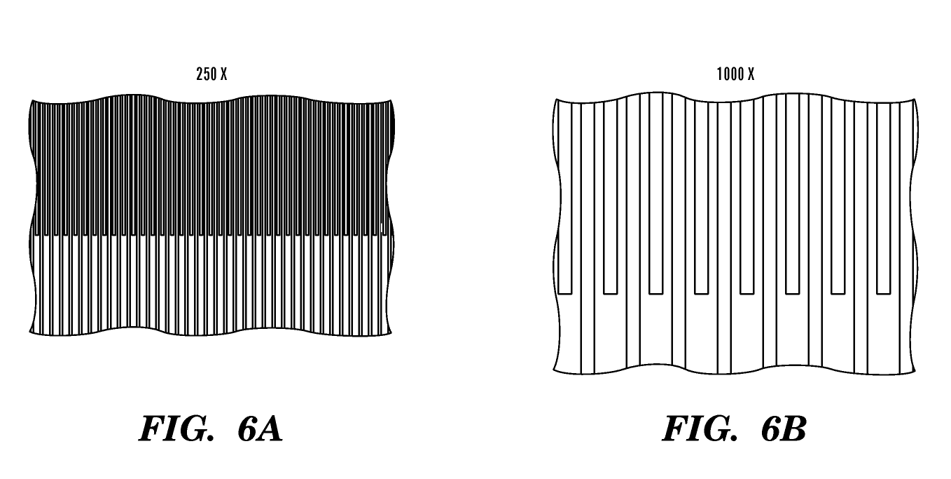

Leak tight sealing was achieved by applying pressure from above PDMS layer 22 and beneath glass plate 26. For this purposes two plates 28 and 4-8 screws 30 were used as shown in FIG. 5. In the case of optical detection, at least the plate 28 adjacent to glass plate 26 is transparent to permit visualization within channel. Alternatively, the optical detection device can be installed between plate 28 and glass plate 26.

It should be noted that the upper plate has tubing 32 and 34 glued into the locations that line up with inlet 102 and outlet 110 of the PDMS device. The pressure applied onto the PDMS-glass plate device also provides a seal for the PDMS-Plexiglas interface. Initially, metal tubing was used in the Plexiglas inlet and outlet holes. However, due to high background signal during the electrochemical detection, these were replaced with plastic tubing.

In order to accommodate a magnet required to capture magnetic beads during the analysis in the capture zone, a groove can be made in the upper Plexiglas plate. The distance between the magnet and the upper wall of the PDMS channel can be precisely controlled by the depth of the groove and the thickness of the PDMS layer. These parameters and the strength of the magnetic field have a great influence on the ability to quantitatively capture beads during the analysis under varying flow rates. The closer the magnet is positioned with respect to the upper wall of the microchannel, the higher flow rates can be used during the analysis. In the microfluidic device of the present invention, the magnet (35DNE1304-NI, Magnet Applications, Inc.) is placed at a distance of 270 μm from the upper wall of the channel. This allows all the beads (1 μm diameter) to be captured at a linear flow rate of 0.2 m/min or 5 μL/min.

The capture device can be any device which achieves non-specific binding (i.e. does not involve use of any of the above-described binding materials). A magnetic field generating device or a filter with a binding material are particularly preferred capture devices. Any suitable solid support can be utilized as the capture support to which the capture device has an affinity. It is particularly preferred to use magnetic beads as the capture support, while the capture device comprises a magnetic field generating device. In this embodiment, as shown in FIG. 1, a paramagnetic particle with a capture probe specific to a target material is contacted with a sample potentially containing the target material under conditions which will permit the target to bind to the capture probe. The resulting complex is then removed from the sample mixture with the magnet. When the filter alternative to a magnetic field generating device is selected, the arrangement of FIG. 5 must be modified so that the filter is in communication with channel 24. Preferably, they can be in a position aligned with the position of magnet 36 (which would not be present in such an embodiment) relative to inlet 102 and outlet 108.

When a filter is used as the capture device, any porous material having a pore size of from about 0.1 μm to about 100 μm, preferably from about 1 μm to about 30 μm, which allows an aqueous medium to flow therethrough can be used. The pore size has an important impact on the performance of the device. The pore size has to be larger than the mean diameter of the marker. Also, the pores should not be too large so that a good volume to surface ratio can be obtained and to hold back the magnetic, polymer, or silica beads coupled to capture probes. Additionally, the filter could function as a conventional filter and retain large particles. Thus, liposomes bound to silica or other particles (the silica or other particles being too large to fit through the filter) would be retained but all other liposomes would pass through the filter. As a result, the amount of target present could be measured by the amount of liposomes bound via target to silica or other particles retained on the filter.

Suitable filter membranes for the device and methods of the present invention include nitrocellulose membranes, nitrocellulose mixed esters, mylar membranes, polysulfonyl based membranes, plain filter paper, glass fiber membranes, and membranes of any plastic material with defined pore size, such as polycarbonate filters, porous gold, and porous magnetic material. It can also be fabricated using microfabrication tools directly inside the microchannel using photoresist materials, such as SU-8 or also PDMS. The filter membranes can be of a variety of shapes, including rectangular, circular, oval, trigonal, or the like.

When the optical detection embodiment of the present invention is utilized, an optical marker is immobilized in the liposome. Suitable optical markers include a fluorescent dye, visible dyes, bio- or chemi-luminescent materials, quantum dots, and enzymatic markers. A qualitative or semi-quantitative measurement of the presence or amount of an analyte of interest may be made with the unaided eye when visible dyes are used as the marker. The intensity of the color may be visually compared with a series of reference standards, such as in a color chart, for a semi-quantitative measurement. Alternatively, when greater precision is desired, or when the marker used necessitates instrumental analysis, the intensity of the marker may be measured directly on the membrane using a quantitative instrument such as a reflectometer, fluorimeter, spectrophotometer, electroanalyzer, etc.

When using liposomes as the particle, the amount of marker material present can be measured without lysis of the liposomes. However, lysis can be used to enhance such visualization. This may be accomplished by applying a liposome lysing agent. Suitable liposome lysing materials include surfactants such as octylglucopyranoside, sodium dioxycholate, sodium dodecylsulfate, saponin, polyoxyethylenesorbitan monolaurate sold by Sigma under the trademark Tween-20, and a non-ionic surfactant sold by Sigma under the trademark Triton X-100, which is t-octylphenoxypolyethoxyethanol. Octylglucopyranoside is a preferred lysing agent for many assays, because it lyses liposomes rapidly and does not appear to interfere with signal measurement. Alternatively, complement lysis of liposomes may be employed, or the liposomes can be ruptured with electrical, optical, thermal, or other physical means.

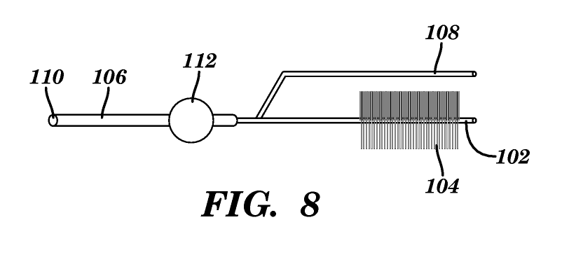

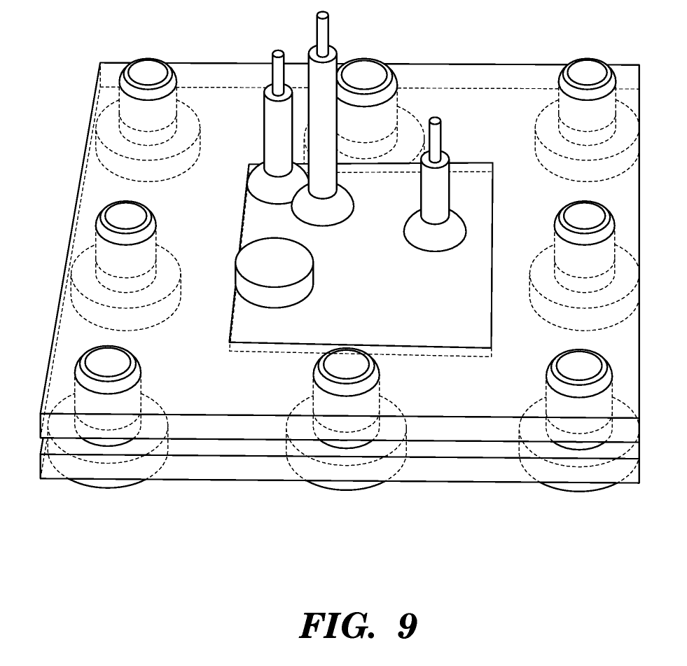

A suitable arrangement for the embodiment of the present invention using optical detection is shown in FIGS. 7 to 8. In operation, as particularly shown in FIG. 8, a test mixture containing a test sample potentially containing the target analyte, a capture conjugate which includes paramagnetic beads, and a marker conjugate is injected through inlet 102. The passage leading from inlet 102 can have a circuitous configuration 104 to provide more residence time for these reactants to contact one another, permitting formation of a product complex which includes the target analyte, the capture conjugate, and the marker. Once the test mixture reaches magnet 112, the product complex is immobilized. Wash liquid is injected into inlet 102 (or 108) which ultimately leads to magnet 112 so that immobilized product complex can be treated to remove unbound marker (e.g., liposomes) which is discharged through outlet 110. If magnet 112 is located in optical detection region 106, such optical detection can take place with the washed product immobilized on magnet 112. Alternatively, whether detection region 106 is located at magnet 112 or downstream of it, the immobilized product complex can be treated such as by agents injected through inlet 108 to release the marker. For example, if the marker is a liposome containing a fluorescent dye, an agent which will disrupt the liposome is injected through inlet 108. As a result, the presence of target analyte in the test sample can be detected by an optical reader. Such detection can also be achieved if detection region 106 is upstream of magnet 112 by causing reverse flow conditions in the channel after marker release.

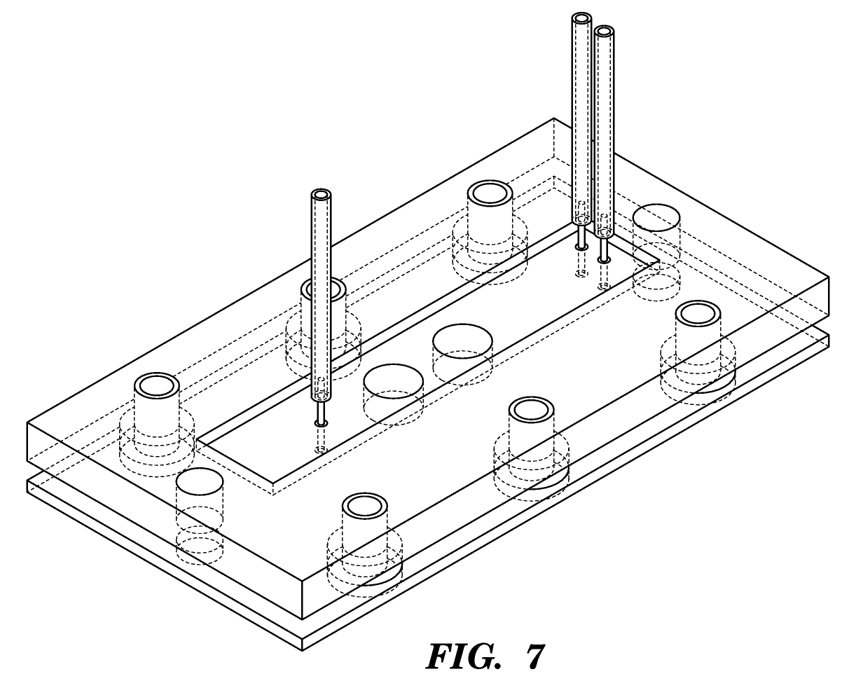

Interdigitated ultramicroelectrode arrays (“IDUA”) can be fabricated on glass wafers using standard photolithographic and lift-off techniques. A typical IDUA was produced by evaporation deposition of 70 nm Ti followed by 500 nm Au on patterned Pyrex glass wafers (7740, Corning, N.Y.). IDUAs with different dimensions were studied as signal transducers in oxidation-reduction reaction of the potassium ferro/ferrihexacyanide, Fe²⁺/Fe³⁺ (CN)₆, pair. It has been shown that both, the background noise and the specific signal depend on the microelectrode's finger/gap ratio as well as on the total amount of fingers (Min et al., “Characterization and Optimization of Interdigitated Ultramicroelectrode Arrays as Electrochemical Biosensor Transducers,” Electroanalysis, 16(9):724-729 (2004), which is hereby incorporated by reference in its entirety). The IDUA designed with 3.8 μm wide fingers and 2.5 μm wide gaps with a total of 1000 electrode fingers demonstrated the best characteristics in terms of sensitivity and signal to noise ratio. Typical microphotographs of IDUAs are present in FIG. 6.

The general principles described above have been used for the device assembling.

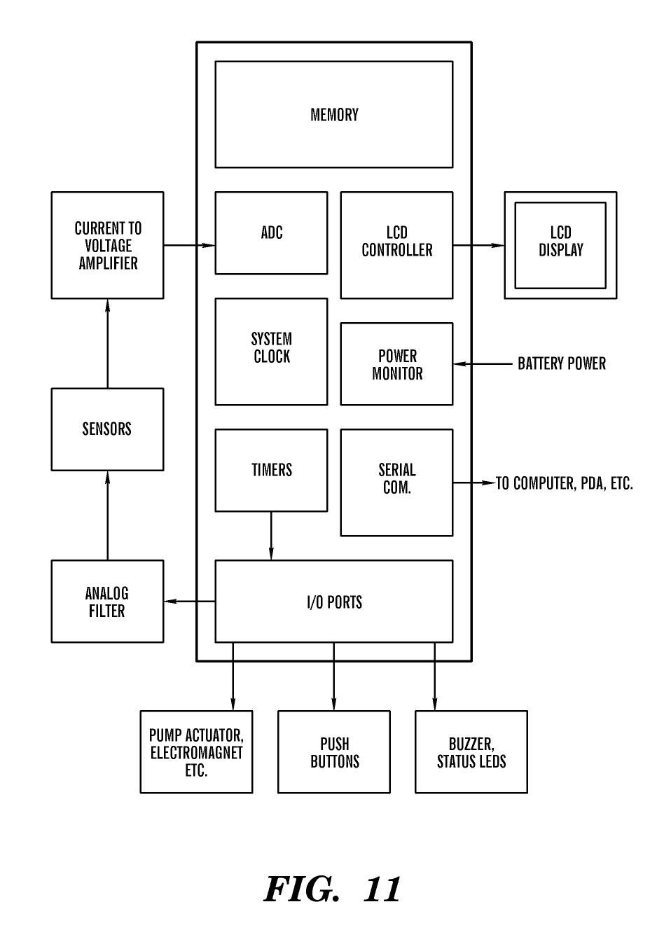

IDUAs fabricated on glass plates are used as signal transducers for the electrochemical signal detection scheme. During assembly, the PDMS channel is positioned on the glass in such a way that the IDUA detection zone is located downstream of the capture zone. In addition, the PDMS channel should be on top of the active microelectrode fingers (FIG. 10).

When the electrochemical detection embodiment of the present invention is utilized, an electroactive species, such as potassiumhexaferrocyanide and potassium hexaferricyanide, is encapsulated in the marker, e.g., liposomes. The microchannel is placed above reusable electrodes, such as an interdigitated electrode array, as described above. After lysis of the liposomes, the quantity of the electroactive species is determined.

Suitable electrochemical markers, as well as methods for selecting them and using them are disclosed, for example, in U.S. Pat. No. 5,789,154 to Durst et al., U.S. Pat. No. 5,756,362 to Durst et al., U.S. Pat. No. 5,753,519 to Durst et al., U.S. Pat. No. 5,958,791 to Roberts et al., U.S. Pat. No. 6,086,748 to Durst et al., U.S. Pat. No. 6,248,956 to Durst et al., U.S. Pat. No. 6,159,745 to Roberts et al., U.S. Pat. No. 6,358,752 to Roberts et al., and co-pending U.S. patent application Ser. No. 10/264,159, filed Oct. 2, 2002, which are hereby incorporated by reference in their entirety. Briefly, the test device may be designed for amperometric detection or quantification of an electroactive marker. In this embodiment, the test device includes a working electrode portion(s), a reference electrode portion(s), and a counter electrode portion(s) in the microfluidic device. The working electrode portion(s), reference electrode portion(s), and counter electrode portion(s) are each adapted for electrical connection to one another via connections to a potentiostat. The test device can instead include a working electrode portion and a counter electrode portion. Alternatively, the microfluidic device may be designed for potentiometric detection or quantification of an electroactive marker. In this embodiment, the device includes an indicator electrode portion(s) and a reference electrode portion(s). The indicator electrode portions and reference electrode portions are adapted for electrical connection to potentiometers. In another embodiment, the test device may include an interdigitated electrode array positioned to induce redox cycling of an electroactive marker released from liposomes upon lysis of the liposomes.

Suitable electroactive markers are those which are electrochemically active but will not degrade the particles (e.g., liposomes) or otherwise leach out of the particles. They include metal ions, organic compounds such as quinones, phenols, and NADH, and organometallic compounds such as derivatized ferrocenes. In one embodiment, the electrochemical marker is a reversible redox couple. A reversible redox couple consists of chemical species for which the heterogeneous electron transfer rate is rapid and the redox reaction exhibits minimal overpotential. Suitable examples of a reversible redox couple include, but are not limited to, ferrocene derivatives, ferrocinium derivatives, mixtures of ferrocene derivatives and ferrocinium derivatives, cupric chloride, cuprous chloride, mixtures of cupric chloride and cuprous chloride, ruthenium-tris-bipyridine, potassium ferrohexacyanide, potassium ferrihexacyanide, and mixtures of potassium ferrohexacyanide and potassium ferrihexacyanide. Preferably, the electrochemical marker is encapsulated within a liposome, in the bilayer, or attached to a liposome membrane surface.

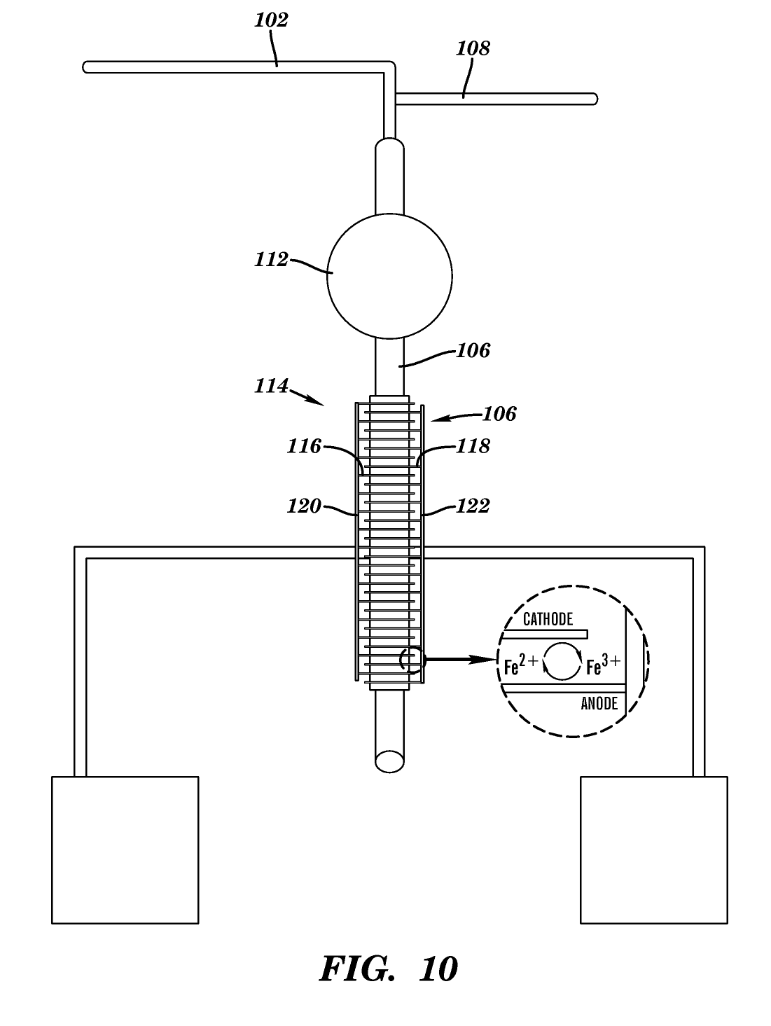

A suitable arrangement for the embodiment of the present invention using electrical detection is shown in FIGS. 9 to 10. In operation, as particularly shown in FIG. 10, a test mixture including a test sample potentially containing the target analyte, a capture conjugate 102. Again, although not shown in FIG. 10, the passage leading from inlet 102 can have a circuitous configuration to provide more residence time for these reactants to contact one another, permitting formation of a product complex which includes the target analyte, the capture conjugate, and the marker. Once the test mixture reaches magnet 112, the product complex is immobilized. Wash liquid is injected into inlet 102 which ultimately leads to magnet 112 so that immobilized product complex can be treated to remove unbound marker (e.g., liposomes). As shown in FIG. 10, when electrical detection region 106 containing IDUA 114 is located downstream of magnet 112, the immobilized product complex can be treated by agents injected through inlet 108 to release the marker. For example, if the marker is a liposome containing a fluorescent dye, an agent which will disrupt the liposome is injected through inlet 108. As a result, the presence of target analyte in the test sample can be detected by the IDUA 114. IDUA 114 is formed from interdigitated fingers 116 and 118 extending from connectors 120 and 122, respectively.

As hereinabove indicated, the assay may be qualitative (presence or absence of certain level of target) or quantitative or semi-quantitative. The preparation of suitable standards and/or standard curves (the term “standard curve” is used in a generic sense to include a color chart) is deemed to be within the scope of those skilled in the art from the teachings herein.

In one embodiment, the test device includes multiple capture portions, each of which is modified to bind a distinctive second binding material specific for one of several analytes. Thus, each analyte may be determined by assignment of each conjugate/analyte to its own measurement portion for concentration and measurement. Alternatively, the conjugate of each of the analytes to be determined in this embodiment of the present invention, may include a marker which is distinctly detectable from the other markers. With different encapsulated dyes (e.g., fluorescent dyes) or quantum dots, the results of the assay can be “color coded”. In particular, a multi-wavelength detector can be used in a capture portion.

As a matter of convenience, the present device can be provided in a kit in packaged combination with predetermined amounts of reagents for use in assaying for an analyte or a plurality of analytes. Included within the kit are stabilizers, buffers, and the like. The relative amounts of the various reagents may be varied widely, to provide for concentration in solution of the reagents which substantially optimizes the sensitivity of the assay. The reagents can be provided as dry powders, usually lyophilized, including excipients, which on dissolution will provide for a reagent solution having the appropriate concentrations for performing the assay. The kit or package may include other components such as standards of the analyte or analytes (analyte samples having known concentrations of the analyte).

As described above, the method and device of the present invention can be used in a variety of assays, such as competitive binding assays and sandwich assays, as described in U.S. Pat. No. 5,789,154 to Durst et al., U.S. Pat. No. 5,756,362 to Durst et al., U.S. Pat. No. 5,753,519 to Durst et al., U.S. Pat. No. 5,958,791 to Roberts et al., U.S. Pat. No. 6,086,748 to Durst et al., U.S. Pat. No. 6,248,956 to Durst et al., U.S. Pat. No. 6,159,745 to Roberts et al., U.S. Pat. No. 6,358,752 to Roberts et al., co-pending U.S. patent application Ser. No. 09/698,564, filed Oct. 27, 2000, and co-pending U.S. patent application Ser. No. 10/264,159, filed Oct. 2, 2002, which are hereby incorporated by reference in their entirety.

Another aspect of the present invention relates to a method for detecting or quantifying an analyte in a test sample, as follows: This method includes providing at least one test mixture which includes a test sample potentially containing an analyte, a capture support complex including a capture support and a first member of a first coupling group, a first binding material selected to bind with a portion of the analyte and including a second member of the first coupling group, a marker complex which includes a particle, a marker, and a first member of a second coupling group, and a second binding material selected to bind with a portion of the analyte other than the portion of the analyte for which the first binding material is selected and including a second member of the second coupling group. The method also involves providing a microfluidic test device for detecting or quantifying an analyte in a test sample. The test device includes a non-absorbent substrate having at least one inlet and outlet extending therethrough, where the inlet and outlet are connected by at least one microchannel imbedded in the substrate, and where the at least one microchannel includes an inlet portion and an analysis portion. The test device also includes a non-specific capture device located at or upstream of the analysis portion. The test device further includes one or more stationary mixing structures extending into the at least one microchannel. Reaction is permitted to occur, within the microfluidic test device, in the at least one test mixture between the first and second members of the first coupling group, between the first and second members of the second coupling group, and between analyte present in the test sample and the first and second binding materials. As a result, a product complex including analyte present in the test sample, the capture support complex, the first binding material, the marker conjugate, and the second binding material is formed. The reacted test mixture is contacted to a non-specific capture device (e.g., a device having non-specific affinity for the capture support) so that product complex present in the reacted test mixture is immobilized from the reacted test mixture. The presence or amount of the marker from the immobilized product complex is detected at the analysis portion of the microfluidic test device and correlated with the presence or amount, respectively, of the analyte in the test sample. In a preferred embodiment, the marker is released from the immobilized product before the contacting and after the detection steps.

The components and steps used to carry out this aspect of the present invention are substantially the same as those described above.

Another aspect of the present invention relates to a method for detecting or quantifying an analyte in a test sample, as follows: This involves providing at least one test mixture including a test sample potentially containing an analyte, a capture conjugate (including a capture support and a first binding material), where the first binding material is selected to bind with a portion of the analyte, and a marker conjugate (including a particle, a marker, and an analyte analog). The method also involves providing a microfluidic test device for detecting or quantifying an analyte in a test sample. The test device includes a non-absorbent substrate having at least one inlet and outlet extending therethrough, where the inlet and outlet are connected by at least one microchannel imbedded in the substrate, and where the at least one microchannel includes an inlet portion and an analysis portion. The test device also includes a non-specific capture device located at or upstream of the analysis portion. The test device further includes one or more stationary mixing structures extending into the at least one microchannel. Competition is permitted to occur, within the microfluidic test device, in the at least one test mixture between analyte present in the test sample and the analyte analog for the first binding material. As a result, a product complex, including the capture conjugate and the marker conjugate, is formed. The reacted test mixture is contacted to a non-specific capture device (e.g., a device having non-specific affinity for the capture support) so that product complex present in the reacted test mixture is immobilized from the reacted test mixture. The immobilized product complex is detected at the analysis portion. The presence or amount of the marker from the immobilized product complex is correlated with the presence or amount, respectively, of the analyte in the test sample. In a preferred embodiment, the marker is released from the immobilized product before the contacting and after the detection steps.

In this embodiment of the present invention, an analyte analog is used, because this embodiment involves a competitive binding assay format. Thus, the term “analyte analog” is meant to include an analog which binds to the capture conjugate. When an analog is employed, however, it is necessary that the particular characteristics of the analyte necessary for recognition by the first binding material in the competition reaction be present in the analyte analog conjugated with the marker complex.

In all other respects, the components and steps used to carry out this aspect of the present invention are substantially the same as those described above.

The present invention also relates to a microfluidic device (also referred to herein as a recirculating microfluidic device, a microfluidic mixing device, or the like). This device includes a non-absorbent substrate having at least one inlet and outlet extending therethrough and one or more stationary mixing structures. The at least one inlet and outlet are connected by at least one microchannel imbedded in the substrate. The one or more stationary mixing structures extend into the at least one microchannel. In all other respects, the components and steps used to carry out this aspect of the present invention are substantially the same as those described above.

In one particular embodiment, the microfluidic device is capable of recirculating microliter volumes. This embodiment of the device can includes molded polydimethyl siloxane (PDMS) channels with pressure inlet and outlet holes sealed by a glass lid. Recirculation is accomplished by repeatedly changing the direction of flow over an iterated sawtooth structure. The sawtooth structure serves to change the fluid velocity of individual streamlines differently dependent on whether the fluid is flowing backwards or forwards over the structure. In this manner, individual streamlines can be accelerated or decelerated relative to the other streamlines to allow sections of the fluid to interact that would normally be linearly separated. Low Reynolds numbers imply that the process is reversible, neglecting diffusion. Fluorescent indicators can be employed to verify numerical simulations. It has been found that mixing of a Carboxyfluorescein labeled DMSO plug with an unlabeled DMSO plug across an immiscible hydrocarbon plug reached steady state in the channels with the sawtooth structures after 7.1 min, versus 34.8 min in the channels without sawtooth structures, which verified what would be expected based on numerical simulations.

### EXAMPLES

### Example 1

### Investigation of Liposome Lysis Using the Fluorescence Detection Approach

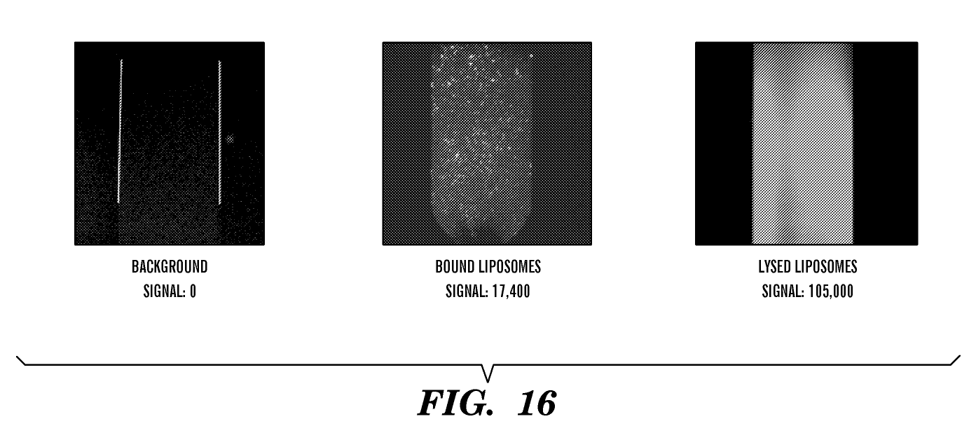

Through inlet 102 of the microfluidic device shown in FIG. 8 and described above, a sample mixture containing complexes of bead—target RNA—liposome is introduced. A mixture containing liposomes encapsulating sulforhodamine B, magnetic beads, target RNA, and a hybridization buffer (60% formamide, 6×SSC, 0.8% Ficoll type 400, 0.01% Triton X-100, 0.15M sucrose) was injected through inlet 102. Captured beads were washed from unbound liposomes by injecting a washing buffer (10% formamide, 3×SSC, 0.2% Ficoll type 400, 0.01% Triton X-100, 0.2M sucrose) into inlet 102. At this point, signals can be detected using the CCD camera connected to the fluorescence microscope. Exposure times were optimized (1 sec), and signals were analyzed using Image Pro Express software. Alternatively, in order to increase the signal to noise ratio by lysing liposomes and obtaining a significantly higher fluorescence signal due to the released sulforhodamine B dye, a solution of 25 mM β-octyl glucopyranoside (OG) was injected into inlet 108 to perform liposome lysis. The fluorescence intensity of the dye released from liposomes was measured by means of a CCD camera connected to the microscope. The device can be operated at preprogrammed volume flow rates from 0.01 to 80 μL/min. Fluorescence of nonlysed (FIG. 16A) and lysed (FIG. 16B) liposomes is detected.

### Example 2

### Optimization of RNA Detection in the Microfluidic Channels

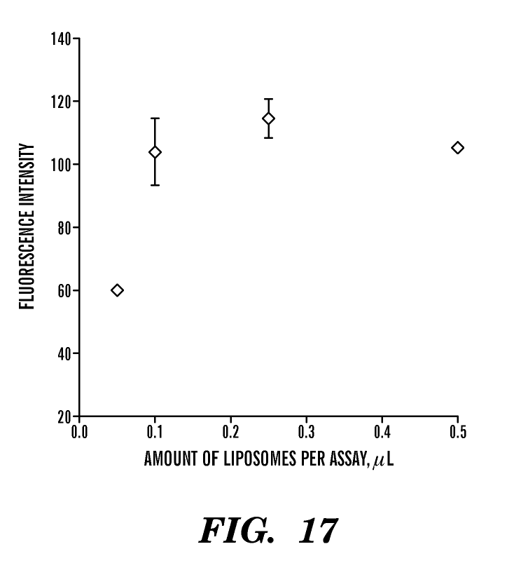

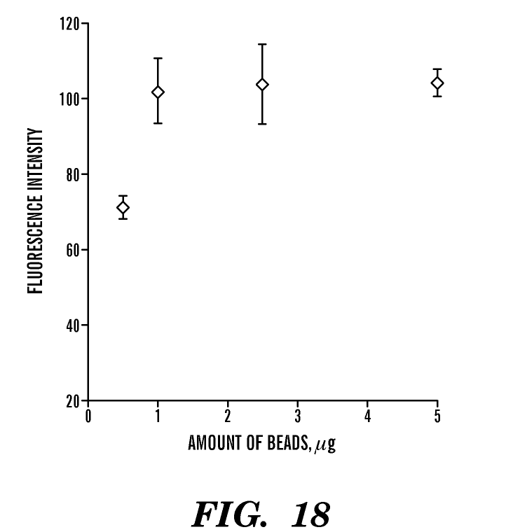

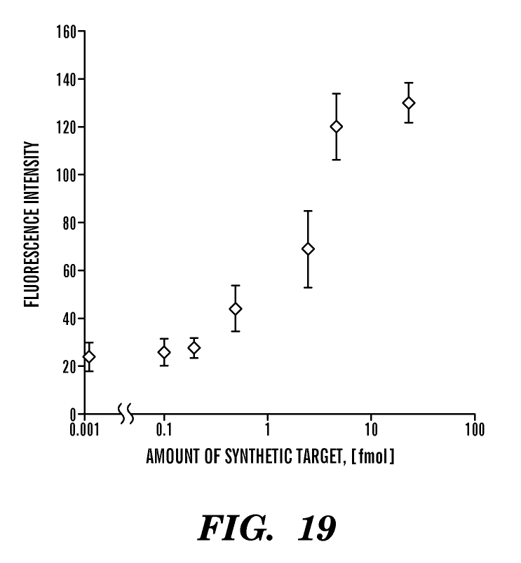

A series of experiments was performed in order to optimize the detection of RNA in the microfluidic channels. These experiments were done without any liposome lysis and were monitored using the fluorescence microscope. The amount of liposomes (1.61 OD value for 1/100 dilution in PBS+ sucrose buffer, pH 7.0, osmolality 630 nmol/kg) with immobilized reporter probe (FIG. 17), beads with immobilized capture probe (FIG. 18) and washing buffer (up to 14 μL) were optimized with respect to signal to noise ratio. Therefore, the limit of detection was obtained for the analysis of Dengue virus RNA. The amount of reporter probe was 0.013 mol % from the total amount of lipids. The biotinylated capture probe was immobilized on the surface of the beads (Dynabeads MyOne Streptavin) following the manufacturer protocol. 1 mg of the beads binds approximately 3,000 pmoles of free biotin.

### Example 3

### Electrochemical Analysis of Liposome Capture in the Microfluidic Device

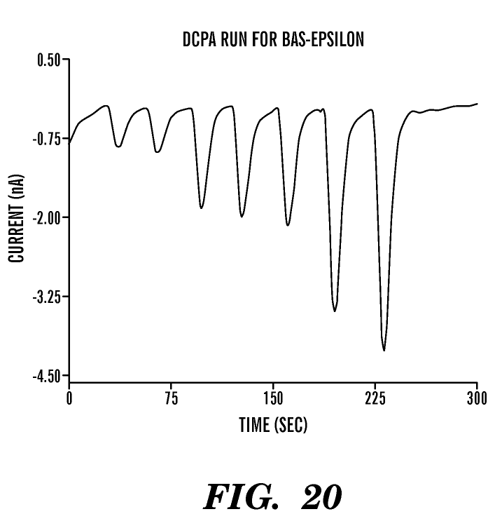

To test the IDUA response in microfluidic system, as shown in FIG. 10, different volumes (20 nL-100 nL) of 10 μM potassium hexaferrocyanide/potassium hexaferricyanide solution were injected at flow rate of 1 μl/min into inlet 108, while buffer solution was introduced at flow rate of 1 μl/min through inlet 102. The typical result of the IDUA response in the single continuous run is provided in FIG. 20.

These results demonstrated that indeed the system based on the IDUA is capable of a fast response to the electrochemical composition changes inside the channel. The delay time between injection and the maximum signal reached was about 5-7 sec. In all the experiments the IDUA itself demonstrates a good reproducibility and the ability to function for prolonged periods of time without mechanical cleaning.

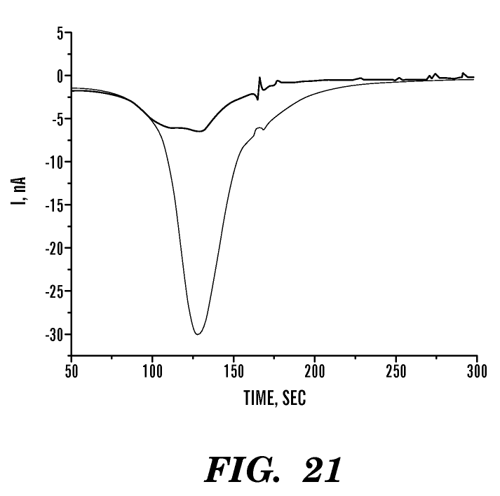

The typical results of RNA analysis by means of electrochemical detection is present in FIG. 21. In this experiment, 2 μl of Dengue serotype 4 amplicon (1:100 dilution) was incubated with 1 μg of supermagnetic beads with attached capture probe and 1 μl of liposomes (150 mM potassium ferro/ferrihexacyanide encapsulant solution).

Hybridization mixture was injected into inlet 102 at 3 μl/min. After all the beads were captured on the magnet and washed with 15 μl buffer, 25 mM solution of OG was injected into inlet 108 at 0.8 μl/min to lyse liposomes. Electrochemical responses of the IDUA in the presence and in the absence of RNA in the hybridization mixture are present in FIG. 21. The signal response of the IDUA to the presence of RNA can be estimated at its peak value or as an integral value of the whole curve (Table 1).

TABLE 1

| | Area | Peak height, nA | Retention time, sec |
|---|---|---|---|
| RNA | 1069 | 28 | 128 |
| Background | 200 | 4.2 | 128 |

A microfluidic biosensor for the highly specific and sensitive detection of pathogens via their nucleic acid sequence has been developed. The biosensor module employs the two alternative methods of detection, fluorescent or electrochemical. A microfabrication approach allows one to use microliter amounts of reagents to perform a single analysis. The microfluidic system was tested and optimized with a model Dengue virus target sequence. It has been shown that as low as 0.5 fmol of the synthetic target can be detected using a microfluidic platform, fluorescence detection method, and nonlysed liposomes.

### Example 4

### Recirculating, Passive Micromixer with a Novel Sawtooth Structure

Experimental data relating to a microfluidic device capable of recirculating nano to microliter volumes in order to efficiently mix solutions is described in this Example 4 and in the below Examples 5-9. The device consists of molded polydimethyl siloxane (PDMS) channels with pressure inlet and outlet holes sealed by a glass lid. Recirculation is accomplished by a repeatedly reciprocated flow over an iterated sawtooth structure. The sawtooth structure serves to change the fluid velocity of individual streamlines differently dependent on whether the fluid is flowing backwards or forward over the structure. Thus, individual streamlines can be accelerated or decelerated relative to the other streamlines to allow sections of the fluid to interact that would normally be linearly separated. Low Reynolds numbers imply that the process is reversible, neglecting diffusion. Computer simulations were carried out using FLUENT (Fluent, Inc.). Subsequently, fluorescent indicators were employed to experimentally verify these numerical simulations of the recirculation principal. Finally, mixing of a carboxyfluorescein labeled DMSO plug with an unlabeled DMSO plug across an immiscible hydrocarbon plug was investigated. At cycling rates of 1 Hz across five sawtooth units, the time was recorded to reach steady state in the channels, i.e., until both DMSO plugs had the same fluorescence intensity. In the case of the sawtooth structures, steady state was reached five times faster than in channels without sawtooth structures, which verified what would be expected based on numerical simulations. The microfluidic mixer is unique due to its versatility with respect to scaling, its potential to also mix solutions containing small particles such as beads and cells, and its ease of fabrication and use.

### Example 5

### Fabrication of Microfluidic Mixer

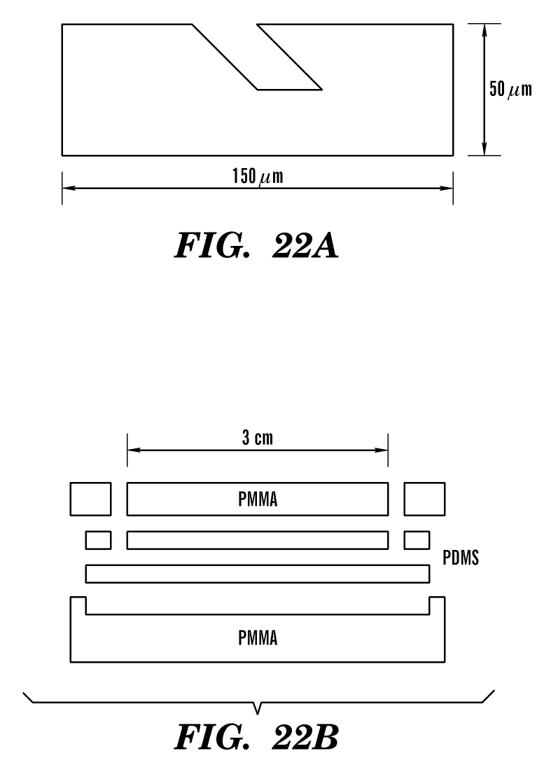

Microfluidic structures were designed using L-Edit (Tanner Research, Inc.) CAD software and fabricated using a silicon master mold and PDMS elastomer (Dow Corning, Corning, N.Y.). The mold was formed by DRIE into a photoresist patterned 100 mm Si wafer at the Cornell NanoScale Science and Technology Facility. After cleaning, Teflon AF (601S1-100-6) was poured, spun, and cured at 170° C. in an oven for 30 minutes. The channels were formed using 7 parts of PDMS elastomer and 1 part curing agent poured over the leveled silicon mold to a thickness of 1 mm and baked at 60° C. for 55 minutes in a vacuum oven at 0.5 bar. After curing, 0.75 mm holes were punched into the PDMS using a cork borer. The PDMS was then sliced into individual channels, oxidized using a Tesla coil, and placed in contact with a cleaned glass lid, where it was left for at least 30 minutes to seal permanently. An acrylic base and lid were used to secure the channels and align them accurately with a set of pressure inlets and outlets (FIG. 22). One inlet was connected to a KD Scientific Model 210 Syringe Pump.

### Example 6

### Mixing Experiments

Carboxyfluorescein was obtained from Sigma-Aldrich Co. DMSO was obtained from Fisher Scientific. Mineral oil was obtained locally. A plug of mineral oil was injected between streams of pure DMSO and 1 mM Carboxyfluorescein labeled DMSO and streams with Carboxyfluorescein labeled DMSO on both sides of the plug, and was visualized using a Leica type DM LB microscope and Coolsnap camera and software package with an exposure time of 1 s using a 300 W UV arc lamp and subsequently color enhanced in Photoshop 7.0 (Adobe Systems, Inc.) using the auto-contrast and auto-level functions only.

### Example 7

### Simulations

Simulations were carried out using FLUENT software (Fluent, Inc.). Meshes were constructed using GAMBIT (Fluent, Inc.). Two-dimensional channels with stationary walls and pressure inlet-outlet ports were simulated. Thirty stream lines across 140 μm of channel length and 50 μm of channel width (at the inlets and outlets, 25 μm at the tip of the sawtooth) were tracked using simulated particle injections. It should be noted that the simulated structure was only one sawtooth unit (for reasons of computational practicality), whereas the experimental device consisted of 200 sawtooth units, each 150 μm long, connected together over a 3 cm channel, with a total volume of approximately 0.5 nL. The same device was fabricated with greater length in order to accommodate about 15 μL of solution.

### Example 8

### Results and Discussion: Recirculating, Passive Micromixer with a Novel Sawtooth Structure

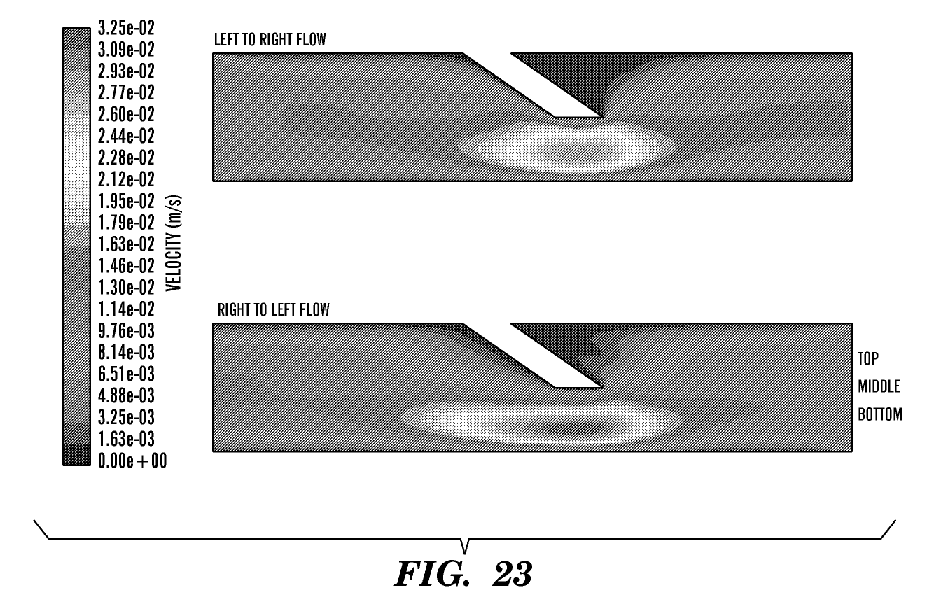

The sawtooth structure of the micromixer was designed to cause mixing of solution in the microchannel based on recirculation. Thus, by repeatedly reciprocating the flow of a solution, some parts of the solution will be relocated with respect to their neighboring volume elements. Mixing occurs by generating transverse flows parallel to the length of the channel, such that streamline segments at different lengths of the channel can be brought into contact with each other. Computational simulations with GAMBIT and FLUENT were used to understand the effects of the sawtooth unit on the flow profiles. FIG. 23 shows the two-dimensional velocity profile of leftward and rightward flows, as viewed from above. The development zone visible in the simulations at the entrance of both channels is due to the model assumption of infinite dimensions outside the channel with a constant flow velocity of 1e-2 m/s. The parabolic flow profile develops approximately 10 μm into the channel, well before the effects of the sawtooth unit must be considered.

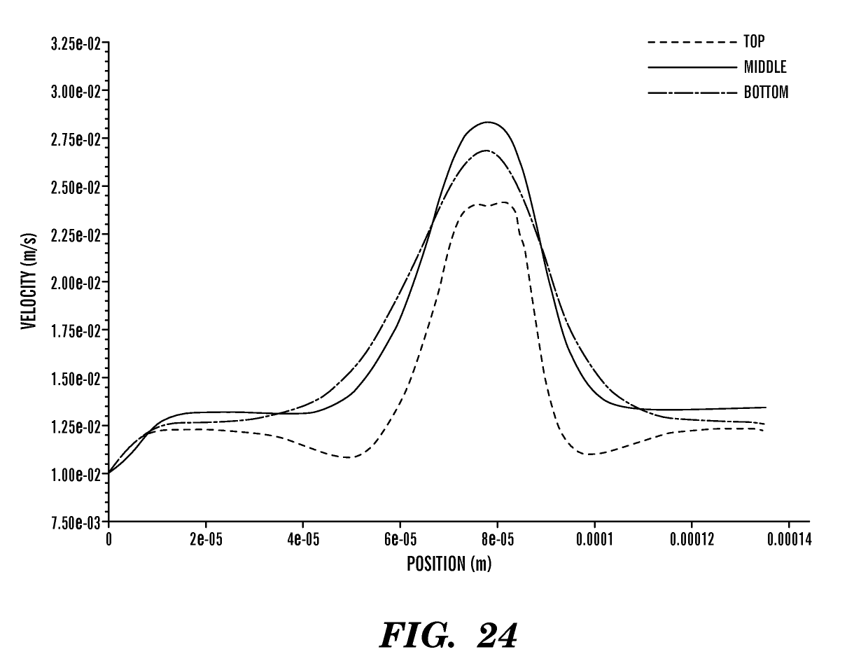

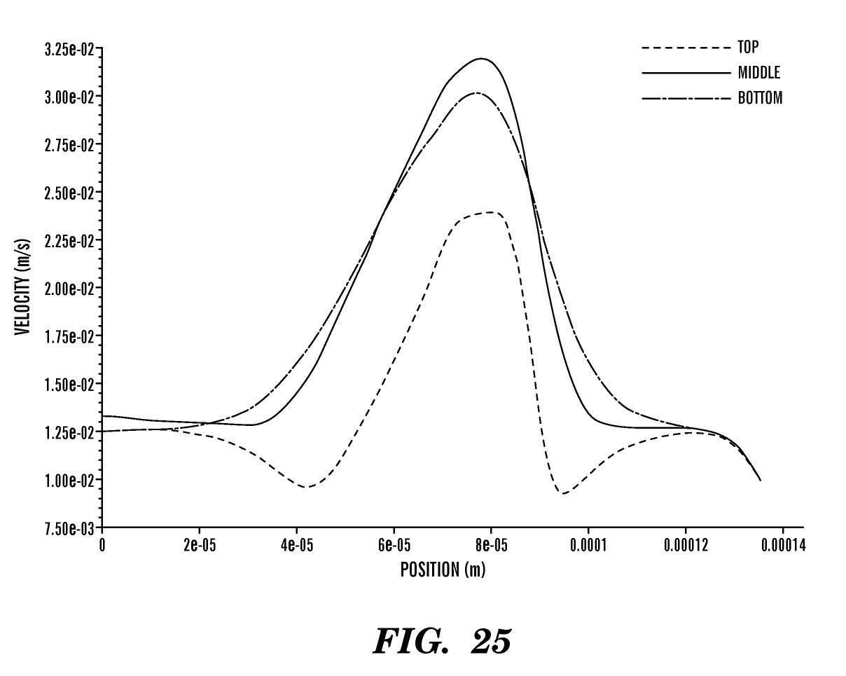

The velocities of the individual streamlines and their profiles are shown in FIGS. 24 and 25. The values of the peak velocity for each streamline are given in Table 2, with the highest velocity streamline (the middle, right to left flow) assigned a value of 100% for comparison.

TABLE 2
Peak velocities for each of the six streamlines
and percent difference between each streamline
and the highest velocity streamline are shown.

| | Peak Streamline | Percent of R-L Middle |
|---|---|---|
| | Velocity [m/s] | Streamline Velocity [%] |
| Right to Left | | |
| Top | 2.4 | 75 |
| Middle | 3.2 | 100 |
| Bottom | 3.0 | 94 |
| Left to Right | | |
| Top | 2.4 | 75 |
| Middle | 2.85 | 89 |
| Bottom | 2.7 | 84 |

The highest velocity streamline was the R to L Middle streamline. This velocity is used as a reference point for comparison with other streamline velocities and thus set to 100%.

Three streamlines were chosen for analyzing the separation efficiency of the streamlines by the sawtooth unit. These three streamlines represent distinct locations in the channel, i.e., in the top quarter (y=0.75*50 μm at x=0 μm), the middle of the channel (y=0.5*50 μm at x=0 μm) and the bottom quarter (y=0.25*50 μm at x=0 μm). The “middle” streamline has the highest velocity due to the parabolic flow profile of pressure driven microfluidic systems. In the left to right flow, a significant decrease of 10% per sawtooth unit in the “bottom” and “middle” streamline velocities was observed compared to right to left flow. It is this difference in rightward and leftward flow profiles that allows for the unique recirculating mixing based on transverse flows parallel to the length of the channel.

Based on the findings of the flow modeling studies, an optimal saw tooth mixer was designed by varying the unit lengths and sawtooth angles. Sawtooth angles were varied in increments of 5° from 20° to 70° and lengths were varied in increments of 2.5 μm from 10 μm to 40 μm. An optimal angle of 45° and length of 25 μm was chosen. The device was subsequently fabricated using standard photolithography and soft-lithography processes. A microfluidic system was assembled consisting of molded PDMS bonded to a glass lid, and connected to a KD Scientific Model 210 Syringe Pump using a machined acrylic assembly as shown in FIG. 22B. PDMS was chosen as the elastomeric material since the feature sizes needed could be realized easily in this material.

A single pump was utilized for the micromixer in order to simplify the requirements of the ultimate design. Thus, positive and negative pressure for the fluid flow were used. By applying a pressure gradient across the entire microchannel, a parabolic flow profile is developed. It was found that devices with unoxidized PDMS leaked under positive pressure flow. Therefore, the surface of the PDMS was modified by oxidation using a Tesla coil to allow for permanent bonding to the glass lid, which created a sufficiently strong bond to allow for both positive and negative pressure to be applied from the same port.

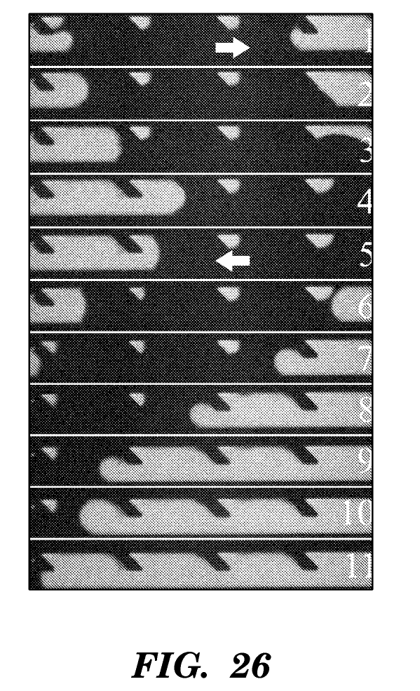

Theoretically, the differences in backpressure between rightward (forward) and leftward (backward) flows that lead to the altered streamline velocity profiles shown in FIGS. 24 and 25 will cause the actual recirculating mixing of solutions. Two experiments were designed to demonstrate this. First, a two-solution system was used in order to demonstrate that solution left behind in the sawtooth structure in the forward pumping direction would be picked up in a different volume location of the fluid during the backward pumping direction. Thus, a system with two plugs of carboxyfluorescein labeled DMSO separated by a plug of hydrocarbons was used. The differences in polarity between the hydrocarbon and the DMSO prevented the two solutions from mixing by diffusion alone. It is recognized that the surface tensions and viscosities of the DMSO/Hydrocarbon plugs are very different from an aqueous solution that will ultimately be mixed in the device. However, the goal was to demonstrate the recirculating mixer principle in a visual manner, and the immiscible plug system was the most convenient for accomplishing this. A typical experiment is shown in FIG. 26, which shows a time lapse image of a hydrocarbon plug moving right, and then left, between two much larger plugs of carboxyfluorescein labeled DMSO. While the recirculation that was theoretically predicted is difficult to observe in this particular image, an alternative form of recirculative mixing can be observed based on the sample held up in the acute angle of the sawtooth structure; fluid is temporarily trapped in this acute angle, and free to diffuse with volumetric elements not originally nearby.

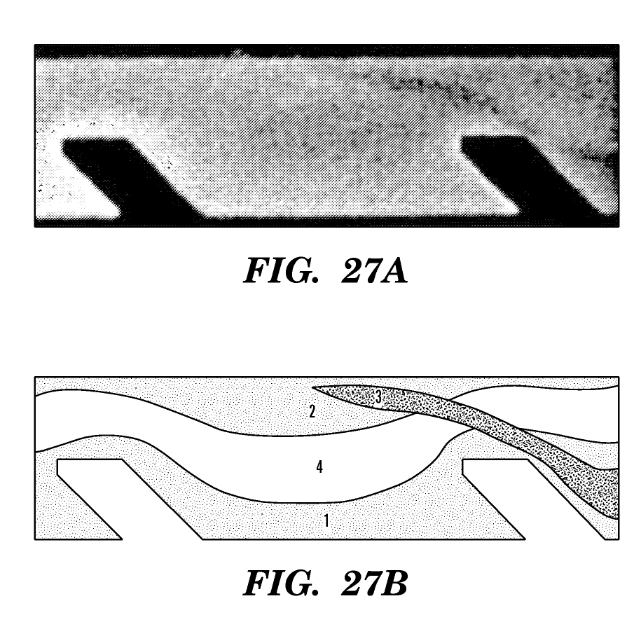

In the second experiment, photographic investigations of the recirculation process were performed in order to prove the presence of recirculation generated by the sawtooth structures by mixing two plugs of DMSO, one fluorescently labeled and one unlabeled each occupying one half of the channel. The fluid was then rapidly moved back and forth (at a set flow rate of 10 μL/min) at a frequency of 1 Hz across the sawtooth structures. During this process photographs were taken of a 200 □m segment (FIG. 27A). At a given time, at least four different intensities of fluorescence could be observed, which correspond to four different concentrations of fluorescent indicator. This is illustrated in FIG. 27B. The presence of these four different concentrations in the same 200 μm window can be best explained by recirculation.

Finally, the mixing efficiency of the micromixer was compared to a straight channel (a channel without sawtooth units) of the same dimensions with respect to channel length, height and width. A hydrocarbon plug was injected between two streams of DMSO, but this time only one DMSO stream was fluorescently labeled. Thus, appearance of fluorescence in the second DMSO plug was again an indication of the recirculation mixing principle based on mixing between different streamlines. The time required for reaching homogeneous fluorescent DMSO plugs of the same fluorescence intensity was an indication of the mixing efficiency. It was determined that diffusion across the solution plug was insignificant by utilizing a plug consisting of unlabeled DMSO/Hydrocarbon/Carboxyfluorescein labeled DMSO left unmoved in the PDMS channel for 48 hours. At the conclusion of the 48 hours, the unlabeled DMSO still showed no fluorescence. In Table 3, typical experimental data obtained are summarized by providing the time needed to reach steady state, which is defined as the amount of time necessary, at continuous mixing of approximately 1 Hz across five sawtooth units (750 μm in the straight channel), for both DMSO plugs to show equal fluorescence.

TABLE 3
Time to Steady State: mixing a plug consisting of unlabeled
DMSO/Hydrocarbon/Carboxyfluorescein labeled DMSO back and
forth across five sawtooth units at approximately 1 Hz.

| | Straight Channel | Sawtooth Channel |
|---|---|---|
| Mean | 34.8 min | 7.1 min |
| Std. Dev | 5.9 min | 1.6 min |

Steady state is defined as the time required for the fluorescence level in the initially unlabeled DMSO plug to equal that of the initially Carboxyfluorescein labeled plug. The “straight” channel was a microchannel of equivalent dimensions to the sawtooth channel, without the sawtooth elements.

### Example 9

### Conclusions: Recirculating, Passive Micromixer with a Novel Sawtooth Structure

Recirculation during mixing is necessary for many microfluidic applications such as enzyme catalyzed reactions, hybridization and binding reactions. It can be readily accomplished using the sawtooth structure described herein. Recirculation was obtained by the structures since these introduce an asymmetry in backward and forward flow that serves to introduce a separation between previously adjacent elements in neighboring streamlines. The fact that recirculation of the solution was obtained was demonstrated via fluid modeling and with three separate experiments. The design can easily be scaled up in length to house microliter volumes, and can bear broader straight channels at either end of the mixer segment to allow all of the solution to pass the entire length of the sawteeth structure. More radical variations on sawtooth placement, such as placing sawteeth on both sides of the channel, using more than one sawtooth length, width and angle in a single channel, etc., can be used. The microfluidic mixer described herein is unique due to its versatility with respect to scaling, its potential to also mix solutions containing small particles such as beads and cells, and its ease of fabrication and use.

### Example 10

### Electrochemical Microfluidic Biosensor

Examples 10-15 relate to experiments regarding the electrochemical microfluidic biosensor and the recirculating microfluidic mixer of the present invention. An electrochemical biosensor for the detection of nucleic acid sequences was developed (Goral et al., “Electrochemical microfluidic biosensor for the detection of nucleic acid sequences,’ Lab on a Chip 6(6):414-421 (2006), which is hereby incorporated by reference in its entirety). To summarize, the target molecule was first hybridized with a capture probe which was immobilized on a paramagnetic bead, and a reporter probe which was conjugated to a liposome. The liposomes encapsulated ferrihexacyanide and ferrohexacyanide, Fe^{2+/3+}(CN)₆. The hybridization solution was then pumped through a 100 μm channel. A magnet was placed on the channel to capture the magnetic beads. Unhybridized liposomes would flow past the magnet and out an outlet port. A solution containing the surfactant octyl glucoside (OG) was then pumped toward the capture zone. Once the OG is in the capture zone, liposomes bound to the magnetic beads via the nucleic acid hybridizations lysed, releasing the redox solution into the channel.

Directly downstream of the capture zone, an interdigitated ultramicroelectrode array (IDUA) was able to measure the current which was proportional to the captured liposome concentration. A dose response curve estimated a limit of detection of 1 fmol of synthetic DNA target. Advantages of this electrochemical detection system include ease of use, cost effectiveness, and portability. Equivalent fluorescent systems require the use of a complicated detection device such as a photomultiplier tube or CCD camera in addition to an excitation source and filters.

### Example 11

### Microfluidic Mixer

Experiments have been conducted on the microfluidic mixer, which can be included in all three modules of the microfluidic device in order to enhance reaction and binding kinetics and avoid diffusion-based limitations. For example, its mixing characteristics, fabrication in larger dimensions (so that NASBA and liposome-RNA binding reactions can be carried out effectively), and fabrication using hot embossing rather than soft lithography were investigated.

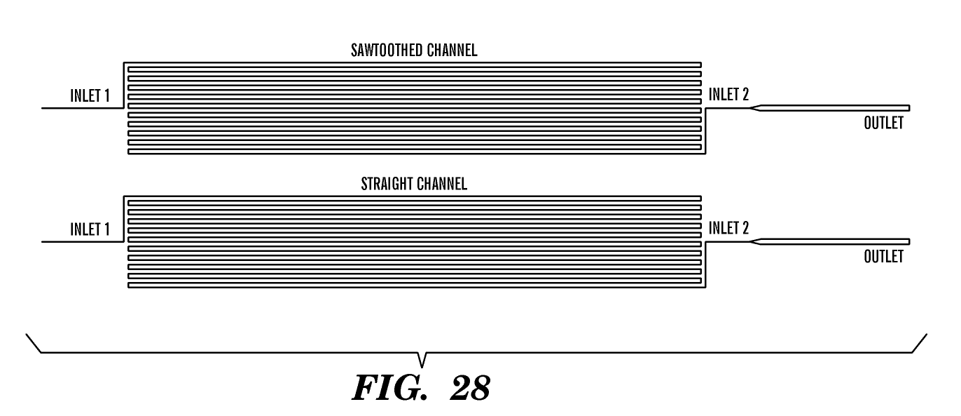

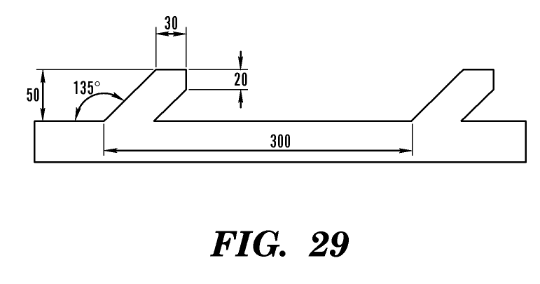

The biosensor can be designed to perform three distinct steps: mRNA isolation, RNA amplification, and RNA detection. A single flow channel pattern was designed to accommodate characteristics needed for all three steps (see FIG. 28). Therefore, three similar channels, with some individual modifications, can be used in series for the detection which will simplify the overall integration into a single device. A channel cross-sectional dimension of 100 μm×100 μm was chosen in order to maintain small sample volumes, resulting in faster loading times. The micromixer investigated here is a key component allowing rapid mixing of solutions and molecules within a solution in a laminar flow regime of the microchannels. A scaled version (FIG. 29) of the sawtoothed micromixer (Nichols et al., “Recirculating, passive micromixer with a novel sawtooth structure,” Lab on a Chip 6(2):242-246 (2006), which is hereby incorporated by reference in its entirety) was designed into a channel with a holding volume of 10 μL. The channel incorporated twenty rows each 5 cm long and containing 166 sawteeth. A detection zone of 500 μm width, was incorporated on the outlet allowing for the placement of an IDUA (Goral et al., “Electrochemical microfluidic biosensor for the detection of nucleic acid sequences, Lab on a Chip 6(6):414-421 (2006), which is hereby incorporated by reference in its entirety) which is only required for the detection step. A second inlet was also incorporated in the detection zone to allow for the introduction of additional reagent during the RNA detection process. The mask design allowed for the etching of two separate devices simultaneously. A channel without sawteeth would be placed adjacent to a channel with sawteeth in order to allow for parallel assays to compare the mixing effect.

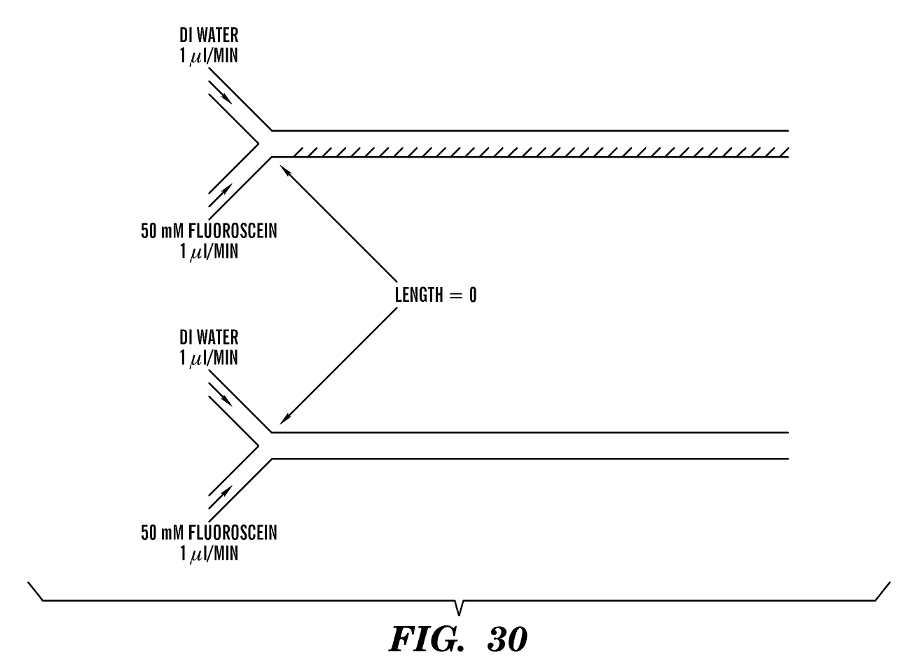

Preliminary work has been done using the original reciprocating mixer using sawtoothed channels with a width of 50 μm using a polydimethyl siloxane (PDMS) body bonded to a glass microscope slide. This channel had two separate inlets and a single outlet (FIG. 30).

One inlet was loaded with DI water while the other inlet was loaded with 50 mM fluorescein. The fluorscein was in a concentration high enough to experience self quenching. Therefore, any dilution of the fluorescein with the DI water would result in an increase in fluorescence during excitation. The flow was controlled using a syringe pump. The flow rate of both inlets was 1 μL/min. The fluorescence across the channel was observed using a microscope mounted with a CCD camera. The pixel intensity was later quantified using Image-Pro Express (MediaCybernetics, Silver Spring, Md.).

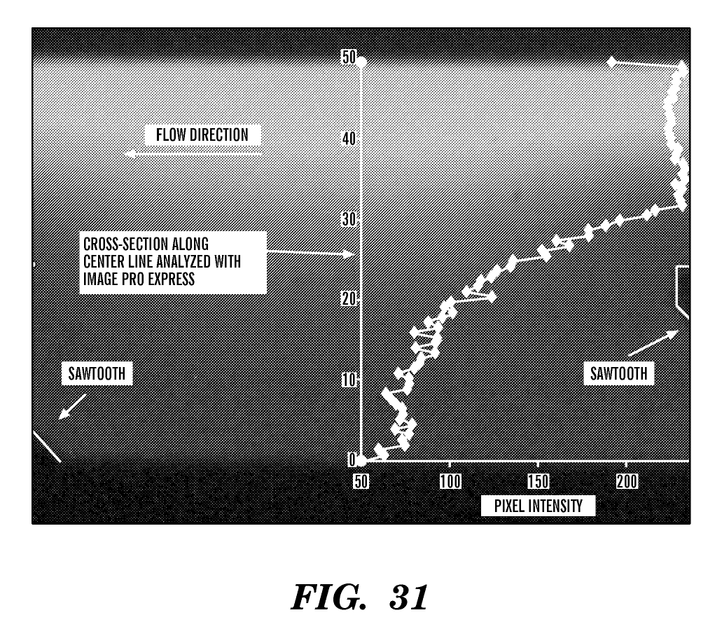

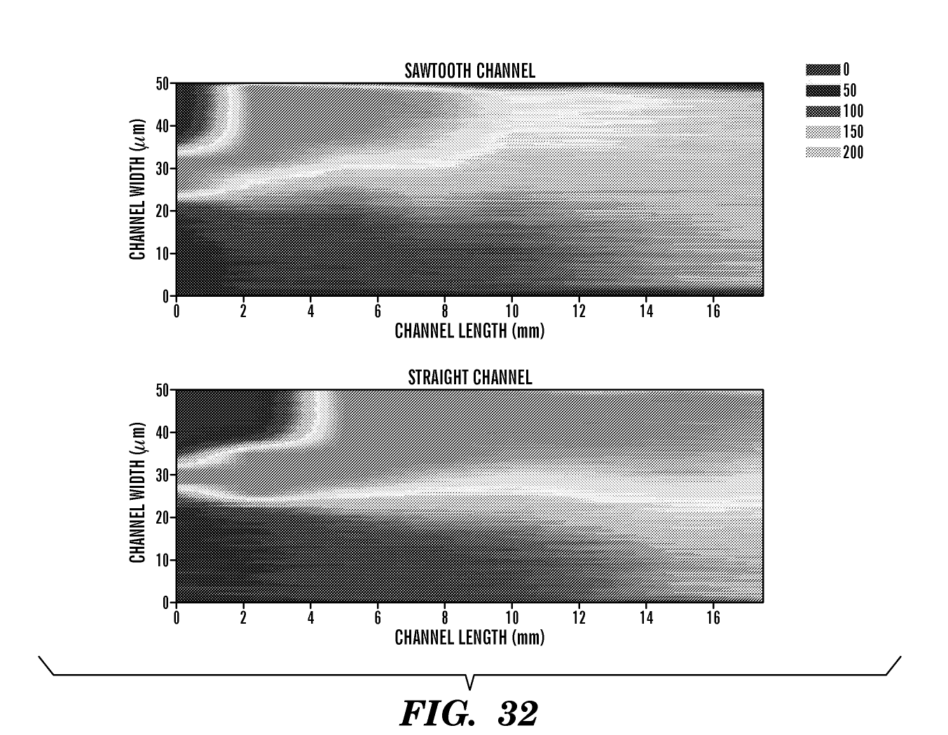

The pixel intensity was measured at the midpoint between the sawteeth for the first two centimeters (FIG. 31). A side by side comparison could then be made between the straight and sawtoothed channel. The measurements on the straight channel were taken at equivalent distances to the sawtoothed channel. The side by side comparison revealed that the sawtoothed structures mixed the solutions in a shorter distance than the straight channel which relies solely on diffusion for mixing (FIG. 32).

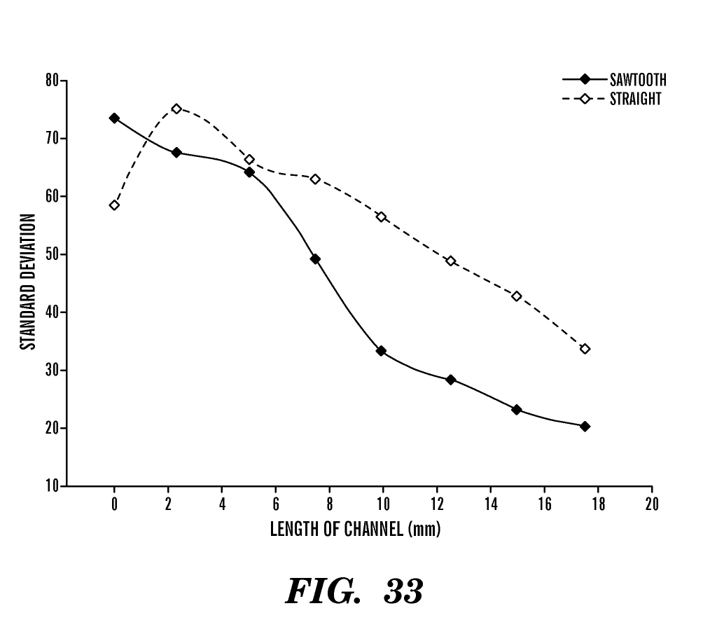

The standard deviation of the pixel intensity across the channel at various distances could also be used as a measure of uniformity of fluorescein concentration (FIG. 33). This comparison demonstrates that the design is effective as a static mixer when compared to a straight channel.

### Example 12

### Microfluidic Device: Photolithography

The device fabrication was performed in part at the Cornell NanoScale Facility (Ithaca, N.Y.). A negative print of a channel was etched on to a silicon wafer using lift off photolithography. Initially, a blank wafer was coated with a layer of primer followed by a layer of S1813 photoresist. After a baking step, the mask was exposed to UV light using a contact aligner (HTG system III-HR, Hybrid Technology Group) for 10 seconds. The overlayed mask allowed exposure only of the areas between the channel structures. The UV light exposed regions become soluble to the photoresist developer. Following a post exposure baking of 90° C. for one minute, the wafer was developed in 1165 developer using an automatic MIF300 to remove the exposed regions of the photoresist resulting in the underlying silicon being exposed.

The wafer was then placed in a Unaxis SLR 770 to etch the channels. Silicon exposed to the inductively coupled plasma/reactive ion environment inside the chamber is etched at a rate of approximately 2 μm per minute. The etching process was allowed to run long enough to obtain a 100 μm deep etch, resulting in a channel height of that depth.

Following etching, the wafer was cleaned of any residual photoresist with acetone. The channel height and width were confirmed using a Tencor P10 profilometer.

### Example 13

### Microfluidic Device: Hot Embossing

The channel patterns were hot embossed into a polymethyl methacrylate (PMMA) substrate using an EV520HE semi-automated hot embossing system. This system allows controlled temperature, a high compression force and a high vacuum. The PMMA is sandwiched between two wafers, the structured wafer on top and a blank wafer below. The sandwich is placed between two temperature controlled plates. The top plate is hydraulically controlled to provide a desired compression force. After the chamber is set to a high vacuum, the top and bottom plates heat to 115° C. before applying 4000 N of force. The high temperature softens the PMMA while the applied force imprints the channel structures into the top of the PMMA. The high vacuum environment ensures that no air bubbles are trapped in the softened PMMA thereby causing channel distortion. The compression is held for 15 minutes before the temperature of both top and bottom compression plates are brought to 100° C. Below the softening temperature, the pressure can be released without disturbing the newly embossed channel structures. After the chamber pressure is normalized the PMMA is removed and the inlet and outlet holes are drilled.

### Example 14

### Microfluidic Device: Bonding

There have been several methods used to bond two pieces of PMMA together while preserving a channel. The most common method is thermal bonding (Yahng et al., “Fabrication of microfluidic devices by using a femtosecond laser micromachining technique and mu-PIV studies on its fluid dynamics,” Journal of the Korean Physical Society 47(6):977-981 (2005); Li et al., “Low-temperature thermal bonding of PMMA microfluidic chips,” Analytical Letters 38(7):1127-1136 (2005); Chen et al., “Vacuum-assisted thermal bonding of plastic capillary electrophoresis microchip imprinted with stainless steel template,” Journal of Chromatography A 1038(1-2):239-245 (2004); and Keynton et al., “Design and development of microfabricated capillary electrophoresis devices with electrochemical detection,” Analytica Chimica Acta 507(1):95-105 (2004), which are hereby incorporated by reference in their entirety), which is hereby incorporated by its entirety), which is hereby incorporated by its entirety), which is hereby incorporated by its entirety). This process uses equipment similar to that used in the hot embossing. Two heated plates heat sandwiched pieces of PMMA until they soften. Under pressure, the softening allows the interface of the two plastics to fuse. This is a fast and simple method of bonding two PMMA pieces. The drawback is the deformation of the channels observed during the process. Another technique used for thermal bonding takes advantage of the ideal bonding temperature being close to 100° C. The two pieces of PMMA are tightly clamped together and immersed in boiling water for one hour (Kelly et al., “Thermal bonding of polymeric capillary electrophoresis microdevices in water,” Analytical Chemistry 75(8):1941-1945 (2003), which is hereby incorporated by reference in its entirety). The advantage is a good heat control. The disadvantage is that commercially available PMMA have different thermal properties depending on the manufacturer. This technique was tested with Optix® PMMA (Plaskolite, Inc., Columbus, Ohio) and found to cause a collapse in the channel due to too high a temperature. The conditions of these bonding techniques would cause the sawtooth structures of the micromixers to distort and lose the intended design. Therefore, a lower temperature technique is needed.

Solvent-assisted thermal bonding involves the application of a very thin layer of solvent on the surface of the unstructured PMMA piece (Klank et al., “CO2-laser micromachining and back-end processing for rapid production of PMMA-based microfluidic systems,” Lab on a Chip 2(4):242-246 (2002), which is hereby incorporated by reference in its entirety). When pressed into the structured piece, the solvent fuses the two pieces together. If this is performed in an 80° C. environment, which is below the thermal distortion range of PMMA not plasticized with solvent, a very uniform seal is made. A spincoater at 4500 rpm for 3 seconds was used in order to have a very thin layer of solvent on the unstructured PMMA. Because the surface of the PMMA is fairly hydrophobic, and most of the solvents are polar, the surface is first treated with O₂ plasma in order to oxidize the surface. This has been shown to increase the hydrophilicity of a PMMA surface.

A lab designed O₂ plasma unit was built using a tesla coil (Model BD-10A Electro-Technic Products Inc., Chicago, Ill.) which is typically used for activation of PDMS by corona discharge as the power source. The point of the tesla coil was placed through a drilled rubber stopper. The stopper was then placed on a PVC cylinder and the pressure in the cylinder was dropped to below 100 mbar. A ten minute treatment was found to be enough to alter the water contact angle from approximately 60 degrees to 42 degrees using a Tantec CAM-Plus (Schaumburg, Ill.) water contact angle meter.

Acetone was not used as a solvent because it was found to be too volatile and was almost completely volatilized after spinning. A higher molecular weight solvent, 2,4-pentadione was found to have ideal volatility properties (Wang et al., “Towards disposable lab-on-a-chip: Poly(methylmethacrylate) microchip electrophoresis device with electrochemical detection,” Electrophoresis 23(4):596-601 (2002), which is hereby incorporated by reference in its entirety). Following plasma treatment, the unstructured PMMA was placed on the spin coater. Enough solvent was placed on the PMMA to completely cover the surface. After 15 seconds, the PMMA was spun at 1,250 rpm for 6 seconds including ramping time. The PMMA was then removed and clamped together with the structures PMMA piece. The clamped pieces were then placed in an 80° C. oven for one hour.

### Example 15

### Microfluidic Device: Device Setup

Once the device has cured, inlet and outlet ports are inserted into the predrilled holes. The tubes were constructed of stainless steel and are held in place by epoxy. The device was again placed in the 80° C. oven for 24 hours to ensure the volatilization of any remaining solvent.

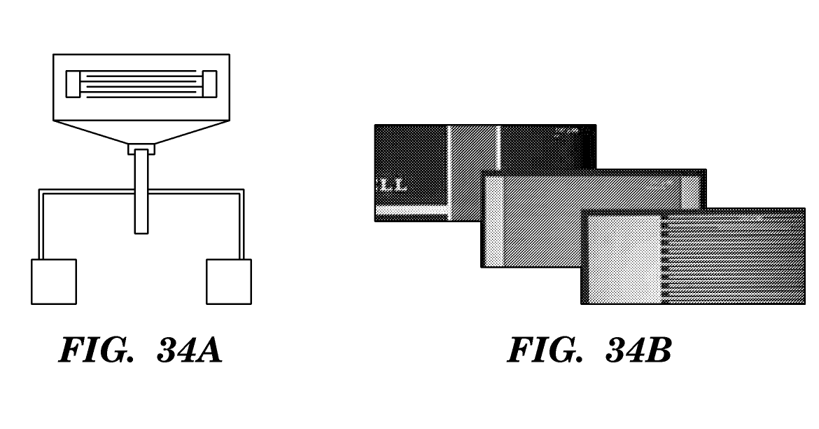

The IDUA will be manufactured using a gold deposition procedure (Goral et al., “Electrochemical microfluidic biosensor for the detection of nucleic acid sequences,’ Lab on a Chip 6(6):414-421 (2006), which is hereby incorporated by reference in its entirety) on a 0.5 mm glass wafer (FIG. 34). Individual IDUAs will be cut from the wafer using a diamond tip dicing saw to a size of approximately 1.6 cm×20 cm. The IDUAs will be used only on the detection devices and not the nucleic acid isolation or NASBA devices.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A microfluidic test device for detecting or quantifying an analyte in a test sample comprising:
a non-absorbent substrate having at least one inlet and outlet extending therethrough, said inlet and outlet connected by at least one microchannel imbedded in the substrate, wherein the at least one microchannel comprises an inlet portion and an analysis portion;
a non-specific capture device located at or upstream of the analysis portion; and
one or more stationary mixing structures extending into the at least one microchannel.

2. The microfluidic test device according to claim 1, wherein said non-absorbent substrate is formed from a material selected from the group consisting of silicon, quartz, glass, polymethylacrylate, polydimethyl siloxane, and polymeric materials.

3. The microfluidic test device according to claim 1, wherein the microchannel further comprises an incubation portion upstream of the analysis portion.

4. The microfluidic test device according to claim 1, wherein said capture device is upstream of the analysis portion.

5. The microfluidic test device according to claim 1, wherein said capture device is at the analysis portion.

6. The microfluidic test device according to claim 1, wherein said capture device is a magnetic field generating device or a filter.

7. The microfluidic test device according to claim 1, wherein the analysis portion comprises an electrochemical detection assembly.

8. The microfluidic test device according to claim 7, wherein the electrochemical detection assembly comprises an electrode array comprising a first conductor having a plurality of fingers and a second conductor having a plurality of fingers, wherein the fingers of the first conductor are interdigitated with the fingers of the second conductor, the first and second conductors are electrically connected to one another via a voltage source and readout device, and the array is positioned to induce redox cycling of the electroactive marker.

9. The microfluidic test device according to claim 7, wherein the electrochemical detection assembly comprises a microcontroller-based analysis system.

10. The microfluidic test device according to claim 1, wherein the analysis portion comprises an optical detection assembly.

11. The microfluidic test device according to claim 1, wherein the at least one microchannel is longitudinally-exposed on a surface of the substrate, said microfluidic test device further comprising:
a cover plate attached to the surface of the substrate and covering the at least one microchannel.

12. The microfluidic test device according to claim 1, wherein there are a plurality of said stationary mixing structures extending into the at least one microchannel.

13. The microfluidic test device according to claim 12, wherein said stationary mixing structures extend different lengths into the at least one micro channel.

14. The microfluidic test device according to claim 12, wherein each microchannel has opposite sides with at least some of said stationary mixing structures extending into the microchannel from the opposite sides in directions generally toward one another.

15. The microfluidic test device according to claim 1, wherein said one or more stationary mixing structures extend into the one or more microchannels at an inclined angle.

16. The microfluidic test device according to claim 15, wherein there are a plurality of said stationary mixing structures with at least some extending into the one or more microchannels at different angles.

17. The microfluidic test device according to claim 1, wherein there are a plurality of inlets to each microchannel.

18. A method for detecting or quantifying an analyte in a test sample comprising:
providing at least one test mixture comprising:
a test sample, wherein the test sample potentially contains an analyte;
a capture conjugate, wherein the capture conjugate comprises a capture support and a first binding material, wherein the first binding material is selected to bind with a portion of the analyte; and
a marker conjugate, wherein the marker conjugate comprises a particle, a marker, and a second binding material, wherein the second binding material is selected to bind with a portion of the analyte other than the portion of the analyte for which the first binding material is selected;
providing a microfluidic test device for detecting or quantifying an analyte in a test sample comprising:
a non-absorbent substrate having at least one inlet and outlet extending therethrough, said inlet and outlet connected by at least one microchannel imbedded in the substrate, wherein the at least one microchannel comprises an inlet portion and an analysis portion;
a non-specific capture device located at or upstream of the analysis portion; and
one or more stationary mixing structures extending into the at least one microchannel;
permitting reaction to occur, within the microfluidic test device, in the test mixture between analyte present in the test sample and the first and second binding materials, thereby forming a product complex comprising analyte present in the test sample, the capture conjugate, and the marker conjugate;
contacting the reacted test mixture with the non-specific capture device, whereby product complex present in the reacted test mixture is immobilized from the reacted test mixture;
detecting the presence or amount of the marker from the immobilized product complex at the analysis portion; and
correlating the presence or amount of the marker from the immobilized product complex with the presence or amount, respectively, of the analyte in the test sample.

19. The method according to claim 18, wherein said permitting reaction to occur and said contacting are carried out by cycling the test mixture in opposite directions in the at least one microchannel.

20. The method according to claim 18, wherein each of the first and second binding materials is an antibody, an antigen, a nucleic acid sequence, an aptamer, or a cell receptor.

21. The method according to claim 18, wherein the analyte is a target nucleic acid molecule, the first binding material is a capture probe selected to hybridize with a portion of the target nucleic acid molecule, and the second binding material is a reporter probe selected to hybridize with a portion of the target nucleic acid molecule other than the portion of the target nucleic acid molecule for which the capture probe is selected.

22. The method according to claim 21, wherein the target nucleic acid molecule is found in an organism selected from the group consisting of bacteria, fungi, yeast, viruses, protozoa, parasites, animals, and plants.

23. The method according to claim 18, wherein the particle is selected from the group consisting of liposomes, latex beads, gold particles, silica particles, dendrimers, quantum dots, fluorescent molecules, dye molecules, and magnetic beads.

24. A method for detecting or quantifying an analyte in a test sample comprising:
providing at least one test mixture comprising:
a test sample, wherein the test sample potentially contains an analyte;
a capture support complex, wherein the capture support complex comprises a capture support and a first member of a first coupling group;
a first binding material, wherein the first binding material is selected to bind with a portion of the analyte, and wherein the first binding material comprises a second member of the first coupling group;
a marker complex, wherein the marker complex comprises a particle, a marker, and a first member of a second coupling group; and
a second binding material, wherein the second binding material is selected to bind with a portion of the analyte other than the portion of the analyte for which the first binding material is selected, and wherein the second binding material comprises a second member of the second coupling group;
providing a microfluidic test device for detecting or quantifying an analyte in a test sample comprising:
a non-absorbent substrate having at least one inlet and outlet extending therethrough, said inlet and outlet connected by at least one microchannel imbedded in the substrate, wherein the at least one microchannel comprises an inlet portion and an analysis portion;
a non-specific capture device located at or upstream of the analysis portion; and
one or more stationary mixing structures extending into the at least one microchannel;
permitting reaction to occur, within the microfluidic test device, in the at least one test mixture between the first and second members of the first coupling group, between the first and second members of the second coupling group, and between analyte present in the test sample and the first and second binding materials, thereby forming a product complex comprising analyte present in the test sample, the capture support complex, the first binding material, the marker conjugate, and the second binding material;
contacting the reacted test mixture with the non-specific capture device, whereby product complex present in the reacted test mixture is immobilized from the reacted test mixture;
detecting the presence or amount of the marker from the immobilized product complex at the analysis portion; and
correlating the presence or amount of the marker from the immobilized product complex with the presence or amount, respectively, of the analyte in the test sample.

25. The method according to claim 24, wherein said permitting reaction to occur and said contacting are carried out by cycling the test mixture in opposite directions in the at least one microchannel.

26. The method according to claim 24, wherein each of the first and second binding materials is an antibody, an antigen, a nucleic acid sequence, an aptamer, or a cell receptor.

27. The method according to claim 24, wherein the analyte is a target nucleic acid molecule, the first binding material is a capture probe selected to hybridize with a portion of the target nucleic acid molecule, and the second binding material is a reporter probe selected to hybridize with a portion of the target nucleic acid molecule other than the portion of the target nucleic acid molecule for which the capture probe is selected.

28. The method according to claim 27, wherein the target nucleic acid molecule is found in an organism selected from the group consisting of bacteria, fungi, yeast, viruses, protozoa, parasites, animals, and plants.

29. The method according to claim 24, wherein the particle is selected from the group consisting of liposomes, latex beads, gold particles, silica particles, dendrimers, quantum dots, fluorescent molecules, dye molecules, and magnetic beads.

30. A method for detecting or quantifying an analyte in a test sample comprising:
providing at least one test mixture comprising:
a test sample, wherein the test sample potentially contains an analyte;
a capture conjugate, wherein the capture conjugate comprises a capture support and a first binding material, wherein the first binding material is selected to bind with a portion of the analyte;
a marker conjugate, wherein the marker conjugate comprises a particle, a marker, and an analyte analog;
providing a microfluidic test device for detecting or quantifying an analyte in a test sample comprising:
a non-absorbent substrate having at least one inlet and outlet extending therethrough, said inlet and outlet connected by at least one microchannel imbedded in the substrate, wherein the at least one microchannel comprises an inlet portion and an analysis portion;
a non-specific capture device located at or upstream of the analysis portion; and
one or more stationary mixing structures extending into the at least one microchannel;
permitting competition to occur, within the microfluidic test device, in the at least one test mixture between analyte present in the test sample and the analyte analog for the first binding material, thereby forming a product complex comprising the capture conjugate and the marker conjugate;
contacting the reacted test mixture with the non-specific capture device, whereby product complex present in the reacted test mixture is immobilized from the reacted test mixture;
detecting the presence or amount of the marker from the immobilized product complex at the analysis portion; and
correlating the presence or amount of the marker from the immobilized product complex with the presence or amount, respectively, of the analyte in the test sample.

31. The method according to claim 30, wherein, said permitting reaction to occur and said contacting are carried out by cycling the test mixture in opposite directions in the at least one microchannel.

32. The method according to claim 30, wherein each of the first and second binding materials is an antibody, an antigen, a nucleic acid sequence, an aptamer, or a cell receptor.

33. The method according to claim 30, wherein the analyte is a target nucleic acid molecule, the first binding material is a capture probe selected to hybridize with a portion of the target nucleic acid molecule, and the second binding material is a reporter probe selected to hybridize with a portion of the target nucleic acid molecule other than the portion of the target nucleic acid molecule for which the capture probe is selected.

34. The method according to claim 33, wherein the target nucleic acid molecule is found in an organism selected from the group consisting of bacteria, fungi, yeast, viruses, protozoa, parasites, animals, and plants.

35. The method according to claim 30, wherein the particle is selected from the group consisting of liposomes, latex beads, gold particles, silica particles, dendrimers, quantum dots, fluorescent molecules, dye molecules, and magnetic beads.

36. A microfluidic device comprising:
a non-absorbent substrate having at least one inlet and outlet extending therethrough, said inlet and outlet connected by at least one microchannel imbedded in the substrate, wherein the at least one microchannel comprises an inlet portion and
one or more stationary mixing structures extending into the at least one microchannel.

37. The microfluidic device according to claim 36, wherein said non-absorbent substrate is formed from a material selected from the group consisting of silicon, quartz, glass, polymethylacrylate, polydimethyl siloxane, and polymeric materials.

38. The microfluidic device according to claim 36, wherein the at least one microchannel is longitudinally-exposed on a surface of the substrate, said microfluidic test device further comprising:
a cover plate attached to the surface of the substrate and covering the at least one microchannel.

39. The microfluidic device according to claim 36, wherein there are a plurality of said stationary mixing structures extending into the at least one microchannel.

40. The microfluidic device according to claim 39, wherein said stationary mixing structures extend different lengths into the at least one microchannel.

41. The microfluidic device according to claim 39, wherein each microchannel has opposite sides with at least some of said stationary mixing structures extending into the microchannel from the opposite sides in directions generally toward one another.

42. The microfluidic device according to claim 36, wherein said one or more stationary mixing structures extend into the one or more microchannels at an inclined angle.

43. The microfluidic device according to claim 42, wherein there are a plurality of said stationary mixing structures with at least some extending into the one or more microchannels at different angles.

44. The microfluidic device according to claim 36, wherein there are a plurality of inlets to each microchannel.

45. The microfluidic device according to claim 36, wherein there are a plurality of outlets to each microchannel.
